(19) United States
(12) Patent Application Publication
(10) Pub. No.: US 2007/0027088 A1
(43) Pub. Date: Feb. 1, 2007

(54) Peptides and mimetics for reducing symptoms of toxic shock syndrome and septic shock

(76) Inventors: John Zabriskie, New York, NY (US); Kumar Visvanathan, Fitzroy (AU)

Correspondence Address: MORGAN & FINNEGAN, L.L.P., 3 WORLD FINANCIAL CENTER, NEW YORK, NY, 10281-2101, US

(21) Appl. No.: 10/555,370

(22) PCT Filed: Mar. 26, 2004
(86) PCT No.: PCT/US04/09450

§ 371 Date: Jul. 12, 2006

Related U.S. Application Data
(60) Provisional application No. 60/458,305, filed on Mar. 28, 2003.

(51) Int. Cl. A61K 38/10; A61K 38/08; C07K 7/08

(52) U.S. Cl. 514/14; 514/15; 530/327; 530/328

(57) ABSTRACT

This invention relates to compositions and methods for providing protection against, or reducing the severity of, toxic shock syndrome, septic shock, food poisoning, and autoimmune diseases which are associated with toxin producing bacteria. This invention also relates to methods of using peptides, derivatives, mimetics, and antibodies for the prevention and treatment of toxic shock syndrome, septic shock, food poisoning, and autoimmune diseases, and other related diseases, conditions, and syndromes.

### FIELD OF THE INVENTION

The present invention relates to compositions and methods for providing protection against, or reducing the severity of, toxic shock syndrome, septic shock, food poisoning, and autoimmune diseases which are associated with toxin producing bacteria.

### BACKGROUND OF THE INVENTION

Shock is a potentially fatal physiological reaction to a variety of conditions, including illness, injury, hemorrhage, and dehydration, usually characterized by marked loss of blood pressure, diminished blood circulation, and inadequate blood flow to the tissues. Toxic Shock Syndrome (TSS) and Septic Shock (SS) are two types of shock that are still among the most life threatening syndromes affecting humans. Toxic shock syndrome (TSS) is a sudden and potentially fatal blood borne condition induced by the release of toxins from bacterium, such as Staphylococcus aureus. Progression of this disease results in a lowering of blood pressure and renal failure. There are approximately 20,000 cases in the United States of TSS each year with a 10% mortality rate (Weiss, K. A. and M. Laverdiere, Can. J. Surg. 40:18-25, 1997). Present therapy is primarily directed at treating symptoms with administration of fluids, antibiotics, vasopressor agents and occasionally steroids (Howe, L. M., Vet. Clin. North Am. Small Anim. Pract. 28:249-267, 1998). There have been numerous vaccine trials for SS, none of which have been successful to date (L. M. Howe. Vet. Clin. North Am. Small Anim. Pract., 28:249-267, 1998; Weiss, K. A., and M. Laverdiere. Can. J. Surg., 40:158-161, 1988).

“Toxic shock like syndrome” is the term previously used to describe the syndromes caused by staphylococcal and streptococcal pyrogenic bacterial exotoxins other than toxic shock syndrome toxin (TSST-1) from S. aureus. Currently, the term “toxic shock syndrome” is used to describe the syndromes caused by TSST-1 and the other bacterial toxins, particularly pyrogenic exotoxins.

Septic shock is another disease which is a condition of shock caused by bacterial endotoxins released in the blood. Septic shock, as used herein, descnbes hypotension and organ failure associated with bacterial infections. In the United States, there are approximately 500,000 reported cases each year, of which 200,000 result in shock with a 40% mortality rate (Schoenberg et al., Langenbecks Arch. Surg. 383:44-48, 1998).

Several clinical features of gram-negative septic shock may be reproduced in animals by the administration of lipopolysaccharide (LPS). The administration of LPS to animals may prompt severe metabolic and physiological changes, which may be fatal. Associated with the injection of LPS is the extensive production of tumor necrosis factor alpha (TNF-α). Mice injected with recombinant human TNF develop piloerection of the hair (ruffling), diarrhea and a withdrawn and unkempt appearance, followed by death if sufficient amounts are given. Rats treated with TNF become hypotensive, tachypneic and die of sudden respiratory arrest (Tracey et al., Science 234, 470474, 1986). Severe acidosis, marked haemoconcentration and biphasic changes in blood glucose concentration were also observed.

Gastrointestinal illnesses may also be induced by bacterial toxins, in particular staphylococcal enterotoxins (Spero and Metzger J., J. Immunol. 120:86-89, 1978). The clinical effect after having ingested only a few micrograms of the toxin occurs in 2 to 4 hours and is manifested by nausea and diarrhea. These symptoms may be caused by leukotrienes and histamine released from mast cells. In addition, both the staphylococcal and streptococcal exotoxins are implicated in gram-positive shock. While superantigen-related septic shock appears to be primarily mediated by TNF-α and IL-12; other cytokines cannot be disregarded (Chapes et al., J. Leukoc. Biol. 55:523-529, 1994; Hackett and Stevens, D. L., J. Infect. Dis. 168:232-235, 1993; Imanishi et al., Int. Arch. Allergy. Immunol. 106:163-165, 1995).

Excessive or unregulated production of TNF has been implicated in mediating or exacerbating a number of diseases including rheumatoid arthritis, rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, reperfusion injury, graft versus host reaction, allograft rejections, fever and myalgias due to infection, such as influenza, cachexia secondary to infection or malignancy, cachexia secondary to human acquired immune deficiency syndrome (AIDS), ARC (AIDS related complex), keloid formation, scar tissue formation, Crohn's disease, ulcerative colitis, or pyresis, in addition to a number of autoimmune diseases, such as multiple sclerosis, autoimmune diabetes and systemic lupus erythematosis.

Toxins which induce TSS, SS, and other related diseases are categorized as endotoxins or exotoxins. Endotoxins are polysaccharide and phospholipid complexes found in the cell walls of primarily gram-negative bacteria and are released upon cell lysis. Endotoxins cause fever and disseminated intravascular coagulation (DIC) defined as widespread coagulation of blood. DIC results in widespread bleeding because blood clotting proteins are exhausted, in addition to low blood pressure and shock, and eventually death if not treated. However, endotoxins are generally weakly toxic and rarely fatal.

Exotoxins comprise a diverse group of soluble proteins released by either gram-positive or gram-negative bacterial cells. Generally, exotoxins are highly toxic and often fatal. Enterotoxins are a subgroup of exotoxins that damage host digestive functions. Staphylococcal enterotoxins have been implicated in staphylococcal food poisoning (Spero et al., J. Immunol. 120:86-89, 1978), as well as toxic shock like syndromes (Bergdoll, M.S. 1985. In J. Jeljaszewicz (ed.). The Staphylococci. Gustav Fischer Verlag, New York, N.Y., pp247-254). For example in food poisoning, Vibrio cholera secretes an enterotoxin that inactivates the Na⁺K⁺ATPase pump of the intestinal epithelial cells, interfering with intestinal cell uptake of nutrients. The toxin thus leads to malabsorption and resulting osmotic diarrhea with water and electrolyte loss.

Group A streptococci pyrogenic exotoxins and the enterotoxins of Staphylococcus aureus, which are also pyrogenic exotoxins, constitute a family of structurally related toxins which share similar biological activities (Hynes et al., Infect. Immun. 55:837-840, 1987; Johnson et al., Molecular General Genetics. 203:354-356, 1986). In addition, the staphylococcal and steptococcal pyrogenic exotoxins also share significant amino acid homology throughout their sequences (Hynes et al. Infect. Immun. 55:837-840, 1987; Marrack and Kappler, Science. 248:705-711, 1990; Hoffman et al., Infection and Immunity. 62:3396-3407, 1994).

In this pyrogenic exotoxin family, there are nine main toxin types, and several allelic variants or subtypes. Many studies have shown that the common motifs shared by the toxins are based on immunologic cross reactivity between the toxins (Spero et al., J. Immunol. 120:86-89, 1978; Spero and Molock, J. Biol. Chem. 253:8787-8791, 1978). These toxins may bind the major histocompatibility complex (MHC) molecules of infected hosts, as well as the variable beta chain (Vβ)of the T cell receptor complex (TCR), causing an aberrant proliferation of specific T cell subsets (Choi et al., Proc. Natl. Acad. Sci. USA. 86:8941-8945, 1989; Fleischer and Schrezenmeier, J. Exptl. Med. 167:1697-1707, 1988; Janeway et al., Immunol. Rev. 107:61-88, 1989). This property of the toxins has labeled them as “superantigens” (White et al., Cell. 56:27-35, 1989) since they do not interact with the MHC and TCR molecules in the manner of conventional antigens (Kappler, et al., Science. 248:705, 1989; Marrack et al., J. Exptl. Med. 171:455-464, 1990).

With respect to superantigens, the Group A Streptococci pyrogenic exotoxins (SPE) and the Staphylococcus aureus enterotoxins (SE) constitute a family which share similar biological activities (Hynes et al., Infect. Immun. 55:837-840, 1987; Johnson et al., Mol. Gen. Gene. 203:354-356, 1986). They stimulate CD4+, CD8+, and γ+ T cells by a unique mechanism. These toxins share the ability to bind the variable beta chain region (Vβ) elements on the lateral face of the T cell Receptor (TCR) and simultaneously bind to the lateral face of the class II major histocompatibility complex (MHC) of antigen presenting cells, causing an aberrant proliferation of specific T cell subsets (Choi et al., Proc. Natl. Acad. Sci. USA. 86:8941-8945, 1989; Fridkis-Hareli, M., and J. L. Strominger, J. Immunol. 160:4386-4397, 1998; Fleischer, et al., Med. Micro. Immunol. 184:1-8, 1995; White, et al., Cell. 56:27-35, 1989). Superantigens have independently evolved over time, and in each case have associated with a different infectious pathogen.

Class II MHC molecules are expressed primarily on cells involved in initiating and sustaining immune responses, such as T lymphocytes, B lymphocytes, macrophages, and the like. Class II MHC molecules are recognized by helper T lymphocytes and induce proliferation of helper T lymphocytes and amplification of the immune response to the particular antigenic peptide that is displayed.

Bacterial toxins, including endotoxins, exotoxins, and enterotoxins, cause a variety of syndromes in humans. Toxic shock syndrome is caused by any of several related staphylococcal exotoxins. The exotoxins of S. aureus are proteinaceous compounds that are secreted at certain times during bacterial growth. The most common TSS toxins are toxic shock syndrome toxin-1 (TSST-1) and staphylococcal enterotoxin B (SEB), where approximately 75% and 20-25% of the cases are caused by these toxins, respectively.

The gene sequences and deduced amino acid sequences of at least six staphylococcal enterotoxins (“SE”): A, B, C, D, E and H, are known, i.e., SEA, SEB, SEC (SEC₁, SEC₂, SEC₃), SED, SEE, and SEH (Marrack and Kappler, Science. 248:705-711, 1990; Reda et al., Infect Immun. 62:1867-1874, 1994). The streptococcal pyrogenic exotoxins (“SPE”) have been implicated in causing the symptoms of scarlet fever and toxic shock like syndrome (Hauser et al., J. Clin. Microbiol. 29:1562-1567, 1991; Merrifield, R. B., J. Am. Chem. Soc. 85:2149-2154, 1963; Stevens et al., The New England Journal of Medicine. 321:1-7, 1989). The sequences of three members of the SPE family: SPEA, SPEC, and SSA, have been reported (Goshorn and Schlievert, P. M., Infect. Immun. 56:2518-2520, 1988; Reda et al., Infect. Immun. 62:1867-1874, 1994; Weeks and Ferretti, J. J., Infect. Immun. 52:144-150, 1986).

Two distinct regions of the SE/SPE toxins that share highly conserved amino acid similarity have been identified. These regions are highly homologous in amino acid sequence and contain consensus patterns which have been identified to be common in all members of this family of toxins (Choi, et al., Proc. Natl. Acad. Sci. USA. 86:8941-8945, 1989). The first consensus region comprises the amino acid sequence Y-G-G-(LIV)-T-x(4)-N. All of the staphylococcal enterotoxins and streptococcal exotoxins except for TSST-1 contain this consensus pattern. The sequence is located at the C-terminal side of the cysteine loop. The second consensus region has the amino acid sequence K-x(2)-(LIV)-x(4)-(LIV)-D-x(3)-R-x(2)-L-x(5)-(LIV)-Y. This particular patter has been identified in all of the staphylococcal enterotoxins, streptococcal pyrogenic exotoxins, including TSST-1 (Bannan, et al., Infect. Dis. Clin. North Am. 13:387-396, ix, 1999; WO 98/45325; WO 00/20598).

TSST-1 shares similar biological activity with the enterotoxins and streptococcal pyrogenic exotoxins; however, it is not as closely related structurally (Blomster-Hautamaa et al., J. Biol. Chem. 261:15783-15786, 1986). Toxic shock syndrome may be exacerbated by the synergistic effects of TSST-1 with the SE/SPE family of toxins (Hensler et al., Infect. Immun. 61:1055-1061, 1993; Smith et al., Infect. Dis. 19:245-247,1994). Stimulation of immune cells by superantigens may aggravate autoimmune syndromes by inducing the expansion of autoreactive T cell subsets, upregulation of MHC-II expression, and the potentiation of cytotoxic T cell response (Brocke et al., Nature. 365:642-644, 1993; Kotzin et al., Adv. Immunol. 54:99-166, 1993; Li et al., Clin. Immunol. Immunopathol. 79:278-287, 1996; Schiffenbauer et al., Proc. Natl. Acad. Sci. USA., 90:8543-8546, 1993; Schwab et al., J. Immunol. 150:4151-4159, 1993).

Specifically, the superantigen, SEB, is capable of inducing toxic shock effects. These effects are the result of activation of a substantial subset of T cells, which lead to severe T cell-mediated systemic immune reactions. This response is characteristic of T cell mediated responses, and may be treated with interleukin 10 (IL-10), or its analogs or antagonists. Mechanistically, the superantigens appear to interact directly with the Vβ element of the T cell receptor and activate T cells with relatively little MHC II class specificity. (See Herman et al., Ann. Rev. Immunol. 9:745-772, 1991).

Although TSS is a specific syndrome caused by either the Staphylococcal or Streptococcal organisms, septic shock is induced by either gram-negative or gram-positive organisms. Lipopolysaccharides (LPS) are an integral part of the cell wall of gram-negative bacteria and are a potent inducer of cytokine released by macrophages (Glauser, M. P., Drugs. 52 Suppl. 2:9-17, 1996). More specifically, LPS binds to macrophage CD14 receptors and triggers the release of several cytolines including IL-1 and TNF-α (Cohen et al., Trends Biotechnol. 13:438445, 1995). Thus, therapies have been designed towards the neutralization of LPS or LPS-induced cytokines (Wang et al., Lymphokine Cytokine Res. 11:23-31, 1992). However, the use of monoclonal antibodies directed against part of the LPS molecule or the use of CD14 soluble receptors in vaccine trials has not resulted in favorable data (Baumgartner, J. D.,Eur J. Clin. Microbiol. Infect. Dis. 9:711-716, 1990). These failures may be a result of: 1) the type of patient selected, where many were already in irreversible shock; 2) the LPS sites were not all blocked by monoclonal antibody; and 3) the LPS molecules were not all blocked by soluble CD14 receptors.

It has been proposed that for lethal septic shock to occur, there must be at least two independent pathways. In fact, there is increasing evidence that both gram-positive infections and gram-negative infections are present in patients with septic shock (Rangel-Frausto et al., JAMA. 273:117-123, 1995). It has also been shown that both LPS and superantigens may act synergistically to produce lethal septic shock in animal models (Schleivert, et al., J. Clin. Immunol. 15:4s-10s, 1995).

Accordingly, a “two hit” hypothesis has been presented, wherein a significant number of septic shock cases involve early gram-negative infection which causes significant symptoms of vasodilation and hypotension. After treatment with fluids and antibiotics, patients quickly recover. However, a few days later, a gram-positive insult either via sepsis of the skin by an intravenous needle or gastrointestinal flora causes severe irreversible shock in a previously LPS-sensitized patient (Bannan et al., Infectious Disease Clinics of North America. 13:387-396, 1996; WO 00/20598). The combined effects of LPS and superantigens significantly increases the lethal properties of both molecules.

In an effort to block the deleterious effects of these toxins, a number of investigators (e.g. Eriksson et al., Microb. Pathog. 25:279-290, 1998; Hu et al., FEMS Immunol. Med. Mircorbiol. 25:237-244, 1999; Jett, et al., Infect. Immun. 62:3408-3415, 1994; Kum, et al., Can. J. Microbiol. 46:171-179, 2000) have used synthetic peptides to block or alter specific actions of known toxins such as staphylococcal enterotoxins B or A (SEB or SEA). Important sites of cytokine production and peptide inhibition for these toxins have been identified. However, only two reports have appeared in which a single peptide is reported to inhibit the proliferative and lethal effects of a number of toxins. One report descnbes using a 12 amino acid peptide CMYGGVTEHEGN (SEQ ID NO: 1) (also called peptide 6343) which is a variant of the native SEB consensus sequence CMYGGVTEHNGN (SEQ ID NO:27) from a common region in order to inhibit blastogenic properties of a large number of toxins. Of these toxins, three new streptococcal toxins not previously described are also reported to be inhibited. Furthermore, this twelve amino acid peptide (6343) was reported to block the lethal effects of three separate and antigenically distinct toxins in a mouse model of toxic or septic shock (Visvanathan, et al., Infection and Immunity. 69:875-884, 2001; WO 00/20598). This 12-mer peptide (6343) binds to a MHC II molecule, which may prevent binding and activation of cell proliferation by superantigens. Arad, et al. use a different 12 amino acid peptide, YNKKKATVQELD (SEQ ID NO:26) (Arad, et al. Nature Medicine 6:414-420, 2000). The peptide of Arad, et al. is a variant of the SEB amino acid sequence 150-TNKKKVTAQELD-161 (SEQ ID NO:29) from a second common region. The peptide reported by Arad, et al. inhibits expression of IL-2 RNA by 18-40 fold and the same results are seen with 2-3 other toxins. In addition, this peptide reportedly rescues mice from the lethal effects of bacterial toxins as observd in a mouse toxic shock model.

Antibodies prepared against peptide regions common to many baterial superantigens have been shown to block the biological effects of several superantigens. For example, antibodies are raised against peptides containing amino acid sequence variants from consensus region 1 (ie., peptide 6344: CMYGGVTEHEGNGC*(SEQ ID NO:23)), consensus region 2 (i.e., peptide 6346: CGKKNVTVQELDYKIRKYLVDNKKLYGC* (SEQ ID NO:24)), and both consensus regions 1 and 2 (ie., peptide 6348: CMYGGVTEHEGNKKNVTVQELDYKIRKYLVDNKKLYGC* (SEQ ID NO:25); where the (*) indicates that the peptides are cross-linked polymers composed of the described sequence). (See, for example, U.S. Pat. No. 6,075,119 and WO 00/20598). Antibodies against peptide 6348 recognize the conserved regions of several bacterial toxin molecules, including TSST-1 (see FIG. 5 of U.S. Pat. No. 6,075,119) and inhibits toxin mediated blastogenesis of human PBMCs stimulated with Staphylococcal and Streptococcal pyrogenic toxins (see FIG. 6 of U.S. Pat. No. 6,075,119). Passive protection against SEB- and SPEA-induced toxic and septic shock in a rabbit model using antibody directed against the peptide is also demonstrated.

However, there still remains a need in the art to provide compositions and methods for preventing and treating individuals having TSS and SS, or other diseases or conditions affected by bacterial toxin.

### SUMMARY OF THE INVENTION

This invention relates to compositions and methods for providing protection against, or reducing the severity of, toxic shock syndrome, septic shock, food poisoning, and autoimmune diseases associated with toxin producing bacteria. This invention also relates to methods of using peptides, derivatives, mimetics, and antibodies (both monoclonal and polyclonal) for the prevention and treatment of toxic shock syndrome, septic shock, food poisoning, and autoimmune diseases, and other related diseases, conditions, and syndromes which result from toxicities associated with bacterial toxins.

An embodiment of this invention relates to peptides, derivatives, and/or mimetics related to homologous sequences of a family of bacterial toxins, including, but not limited to staphylococcal and streptococcal pyrogenic toxins, antibodies thereto, and compositions thereof.

In one embodiment of this invention, peptides, derivatives, and/or mimetics thereof have the amino acid sequence comprising one tyrosine residue in a dimer or trimer, and/or a contiguous methionine and tyrosine amino acid sequence, and/or TEHEGN (SEQ ID NO: 7) amino acid sequence, wherein said peptide, derivative, and/or mimetic consists essentially of 12 or fewer amino acids, with the proviso the amino acid sequence is not an amino acid sequence which is found in any native toxin molecule and the peptide is not CMYGGVTEHEGN of SEQ ID NO: 1 or any peptide specifically disclosed in U.S. Pat. No. 6,075,119 and WO 98/45325 which are both incorporated herein by reference.

A further embodiment of the invention provides modified peptides, derivatives, or mimetics, wherein said peptides, derivatives, or mimetics that are in the natural L-conformation, or more preferably the D-conformation. In particular, one embodiment of the invention relates to D-conformation trimers, cmy and ymc. Yet another embodiment of the invention relates to dimers or trimers comprising a contiguous methionine and tyrosine, such as but not limited to, L-conformation peptides, AMY, MYC, CYM, and MY. In a further embodiment, dimers or trimers containing a tyrosine, such as but not limited to, CAY and CY are also provided. Longer peptides comprising any of these functional dimers or trimers are also provided.

One embodiment of the invention provides isolated and purified nucleic acids encoding the peptides, derivatives, or mimetics of the invention, as descnbed herein, and transformed host cells containing these nucleic acids.

Another embodiment of the invention provides pharmaceutical compositions comprising a peptide, derivative, mimetic, or antibody as described herein, or a structurally and/or immunologically-related antigen in a pharmaceutically- and physiologically-acceptable carrier for the prevention and treatment of toxic shock syndrome, septic shock, food poisoning, autoimmune diseases, and other related diseases, conditions, and syndromes.

The invention further relates to the use of these compositions in diagnostic assays and in prophylactic and therapeutic methods for preventing or treating toxic shock syndrome, septic shock, food poisoning, autoimmune diseases, and other diseases, syndromes, and conditions which result from toxicities associated with bacterial toxins.

In one embodiment of this invention, methods of inhibiting blastogenesis of human mononuclear cells in the presence of at least one bacterial toxin by administering a peptide, derivative, or mimetic of this invention are provided.

It is a preferred embodiment of the invention to treat an individual at risk for developing toxic shock syndrome, septic shock, food poisoning, or autoimmune diseases associated with bacterial toxins, or an individual with symptoms of toxic shock syndrome, septic shock, food poisoning, or autoimmune diseases associated with bacterial toxins by administering to such individual the peptides, derivatives, or mimetics of this invention.

One embodiment of this invention relates to methods of passive immunization of a mammal against the toxic effects of bacterial toxins by administering in vivo, an immunologically sufficient amount of an antibody which binds to a peptide, derivative, or mimetic and at least one bacterial toxin.

A further embodiment of the invention provides methods of inducing antibodies that bind at least one bacterial toxin by administering a peptide, derivative, mimetic, or nucleic acid encoding at least one peptide, of this invention, and methods of their use for preventing, treating, or protecting against the toxic effects of bacterial toxins, including, but not limited to most, if not all, of the staphylococcal and streptococcal pyrogenic toxins.

Further, in another embodiment of the invention, methods of detecting antibodies to bacterial toxins in a sample are provided, where the sample is contacted with a peptide, derivative, or mimetic of this invention and the peptide, derivative, or mimetic bound to the antibody is detected.

Also, an embodiment of the invention provides diagnostic assays and kits comprising peptides, derivatives, kimetics, and/or antibodies against the peptides, derivatives, or mimetics for detecting the presence of bacterial toxins.

### BRIEF DESCRIPTION OF THE FIGURES

FIG. 1 shows the results of algorithmic alanine substitution at specific locations of the peptide versus SEB, as determined by blastogenesis assay measuring incorporated tritiated thymidine and using human PBMCs. Peptide constructs in which single alanine amino acid substitutions of the entire 12-mer peptide (6343) are prepared and added to the wells (Table 1). Substitution of alanine at any of the first three amino acids of the N-terminal end of the peptide results in the greatest loss of inhibitory activity of the peptide.

FIG. 2 shows the average results of blastogenesis assays using peptide derivatives constructed to have amino acids deleted from the N-terminal and C-terminal ends (Table 2). The graph shows a comparison in percent inhibition of the various peptide derivatives to SEB (2 μg/well), as measured by incorporated tritiated thymidine. NM represents normal media and PHA is phytohemagglutinin, a positive mitogenic control.

FIG. 3 shows the results of blastogenesis assays with human PBMCs. The graph shows a comparison in percent inhibition of peptides: CMY, acetylated CMY, cmy, YMC, ymc, and 12-mer peptides 6343 and 6343S to SEB (2 μg/well) by measuring incorporated tritiated thymidine. A scrambled version of peptide 6343 is the 6343S peptide (EHEGNCMYGGVT; SEQ ID NO:28). Each of the peptides are added in varying doses ranging from 25 μg to 200 μg, as indicated. Capital letters indicate the natural L-conformnation of the peptide while lower case letters denote the D-conformation of the peptides. The 6343S peptide has a minimal effect (less than 20%) on inhibition of SEB proliferation. CMY or YMC are almost as effective as the original 12-mer peptide 6343 in blocking superantigen activity. Surprisingly, the D-conformations, cmy and ymc, are the most effective in blocking superantigen activity.

FIG. 4 shows the results of blastogenesis assays using human PBMCs as described above and in Example 1. The graph shows the comparison in percent inhibition of various peptide derivatives and 12-mer peptide (6343) to SEB (2 μg/well), as measured by incorporated tritiated thymidine. Various combinations of dimer and trimer peptides containing tyrosine (Y) are all effective. Lower doses of these dimers and trimers are also more effective than the 12-mer peptide.

FIG. 5 shows the blastogenesis assay results using human PBMCs and SEB toxin according to methods described above and in Example 1. The peptides are added to cells in varying doses ranging from 75 μg-200 μg. Although dimers and trimers containing tyrosine are found to be especially effective (FIG. 4), the trimer peptide containing three tyrosines (YYY) is not inhibitory.

FIG. 6 shows the comparison in percent inhibition of peptide derivatives: CMYGK (SEQ ID NO: 21) and CMYKK (SEQ ID NO: 22) and 12-mer peptide (6343) compared to SEB (2 micrograms/well) at various concentrations of peptide as measured by incorporated tritiated thymidine. All three peptides result in similar inhibitory percentages.

FIG. 7 shows the results of a peptide blastogenesis assay comparing peptides: CMY, CMYG (SEQ ID NO: 20), and 12-mer peptide (6343) compared to SEB (2 micrograms/well) at varyious concentrations of peptide as measured by incorporated tritiated thymidine. The trimer has similar inhibitory effects as compared to peptide 6343.

FIG 8 shows results of a blastogenesis assay using human PBMCs and SEB toxin. Cells (100 microliters) are mixed with either PHA (5 micrograms) in the well alone or with 200 micrograms of peptide (CMY, YMC, ymc, and cmy) for a total volume of 200 microliters in the well. After 96 hours incubation, 3 microCuries of tritiated thymidine is added to each well. The cell mixture is further incubated for 18 hours and then collected for analysis by using a beta count reader. There is no significant difference in the PHA stimulation counts with or without peptides.

### DETAILED DESCRIPTION OF THE INVENTION

This invention relates to compositions and methods for providing protection against, or reducing the severity of shock, including but not limited to, toxic shock syndrome, septic shock, food poisoning, and autoimmune diseases associated with toxin producing bacteria.

In particular, this invention provides a class of peptides useful for preventing or treating toxicity due to bacterial infection. Peptides or derivatives thereof, of this invention consisting essentially of 2-12 amino acids, preferably 2-9 amino acids, more preferably 2-6 amino acids, and most preferably 2-3 amino acids. The peptides or derivatives of this invention have one tyrosine residue in a dimer or trimer, and/or a contiguous methionine and tyrosine amino acid sequence, and/or TEHEGN (SEQ ID NO: 7) amino acid sequence. The peptides of this invention are not the 12-mer peptide 6343 having the amino acid sequence CMYGGVTEHEGN (SEQ ID NO:1), or any of the peptides specifically disclosed in U.S. Pat. No. 6,075,119 and WO 98/45325 which are both incorporated herein by reference.

Peptides of this invention include any substituted analog or chemical derivative or mimetic thereof, of a peptide that is related to the consensus sequence of staphylococcal enterotoxins (SE) and streptococcal pyrogenic exotoxins (SPE). Preferred peptides of this invention inhibit toxin-mediated blastogenesis and block toxin activity by approximately 45-50%, preferably 50-60%, more preferably 60-80%, and most preferably, 80-100%. Non-limiting examples of peptides of this invention have amino acid sequences of: CY, MY, CMY, CYM, YMC, MYC, AMY, CAY, CMYGGVTEHEG (SEQ ID NO:4), CMYGGVTEHE (SEQ ID NO:5), CMYGGV (SEQ ID NO:6), TEHEGN (SEQ ID NO:7), CMYAGVTEHEGN (SEQ ID NO: 11), CMYGAVTEHEGN (SEQID NO: 12), CMYGGATEHEGN (SEQ ID NO:13), CMYGK (SEQ ID NO:21), and CMYKK (SEQ ID NO:22), where the peptide, or derivative thereof, is in its natural L-conformation, and preferably in its D-conformation. Furthermore, a longer peptide comprising the peptides of 2 -6 amino acids is also contemplated in an embodiment of the invention. Peptides of the invention are preferably not toxic, but toxic peptides may be useful in this invention, for example, in eliciting antibodies in a non-human system. Paricularly preferred are those peptides that, surprisingly, consist of only two or three amino acids, and more preferably, D-conformational dimers and trimers, and that maintain their inhibitory effects.

Unexpected results show that peptides possessing relatively few amino acids, such as dimers and trimers, are as effective, if not more, than longer peptides for reducing or inhibiting toxicity associated with bacterial toxins or bacterial toxins themselves. Thus, this invention includes peptides consisting essentially of six amino acids, preferably five amino acids or four amino acids, and more preferably peptides of only three amino acids or two amino acids are also effective in inhibiting bacterial toxin. Without being bound by theory, it is believed that the peptides, derivatives, mimetics, and/or antibodies directed against the peptides, of the present invention block the toxin pathway, thereby preventing the onset of bacterial toxin poisoning, and in particular, lethal shock induced by the combination of LPS with one or more superantigens.

In particular, an embodiment of this invention provides compositions comprising peptides, derivatives, and/or mimetics thereof related to a conserved region of bacterial toxins, preferably, staphylococcal enterotoxins and streptococcal pyrogenic toxins. These compositions are useful for providing protection against, or reducing the severity of bacterial induced shock, such as toxic shock syndrome, septic shock, autoimmune reactions, and food poisoning from bacterial infections, in mammals, including humans.

A further embodiment of the invention relates to the peptides themselves, or as used as haptens, are capable of eliciting the production of antibodies which can bind to bacterial toxins, specifically, the staphylococcal and streptococcal pyrogenic exotoxins, or endotoxins. Antibodies generated according to this invention using the peptides described herein also bind to staphylococcal and streptococcal pyrogenic exotoxins.

Definitions

A “peptide” of this invention refers to any substituted analog or chemical derivative of an isolated and purified peptide. The term “peptide” as used herein, should also be construed as referring to any amino acid sequence of any molecular weight, or chemical derivative thereof. The term “derivative” as used herein is a substance related to another substance, such as a peptide, by modification or partial substitution.

Peptides of this invention, derivatives, or mimetics thereof, also include, but are not limited to those amino acid sequences that are altered, in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, non-polar (hydrophobic) amino acids include alanine, leucine, isoleucine, valine, proline, phenylalanine, tryptophan, and methionine. Polar neutral amino acids include glycine, serine, threonine, cysteine, tyrosine, asparagine, and glutamine. Positively charged (basic) amino acids include arginine, lysine, and histidine. Negatively charged (acidic) amino acids include aspartic acid and glutamic acid.

The invention further relates to “mimetics,” defined herein as molecules which mimic elements of protein secondary structure, such as the peptides described in this invention. See, for example, Johnson et al. (In: Biotechnology and Pharmacy, Pezzuto et al., (Eds.), Chapman and Hall, New York, 1993). In particular, minimetics or peptidomimetics are sterically similar compounds that are formulated to mimic the key portions of the protein or peptide structure or to interact specifically with the MHC. The design of “mimetics” can involve arranging functional groups such that the functional interactions between molecules are reproduced. Mimetics may be desirable when the active compound is either difficult or expensive to synthesize, or when administration of an active compound is ineffective, e.g. peptides for oral compositions are quickly degraded by proteases in the alimentary canal (U.S. Pat. No. 5,770,377 to Picksley, et al.). In particular, these mimetics are directed to, but are not limited to, the peptides or derivatives of this invention that inhibit toxin activity and blastogenesis of the toxins.

The present invention describes mimetic molecules that contact the alpha1 domain alpha helix of the MHC class II molecule. Since peptides and mimetic molecules suppress or block interaction between superantigens and MHC class II molecules, superantigen activity and blastogenesis of the toxins is also inhibited. Also encompassed by the present invention are molecules which mimic or imitate the hydrophobic interaction(s) between the alphal domain alpha helix of the MHC class II molecule and the bacterial toxin, such as but not limited to the superantigen, SEB. Such mimetic molecules possess structural similarity molecules having hydrophobic interactions with the alphal domain alpha helix of the MHC class II molecule. Non-limiting examples of mimetic molecules are described herein and have similar properties to or contain any one of the following amino acid sequences: CY, MY, CMY, CYM, YMC, MYC, AMY, CAY, CMYGGVTEHEG (SEQ ID NO:4), CMYGGVTEHE (SEQ ID NO:5), CMYGGV (SEQ ID NO:6), TEHEGN (SEQ ID NO:7), CMYAGVTEHEGN (SEQ ID NO: 11), CMYGAVTEHEGN (SEQID NO: 12), CMYGGATEHEGN (SEQ ID NO: 13), CMYGK (SEQ ID NO:21), and CMYKK (SEQ ID NO:22).

The mimetic molecules of the invention are amino acid sequences, peptides, polypeptides, or small molecules, synthetic or natural organic products, which share structural similarity with a native ligand, such as a toxin, for the MHC class II molecule alpha1 alpha helix containing polypeptide and interacts with the alpha1 domain alpha helix containing polypeptide and thus modulates the activity of the alpha1 domain alpha helix containing polypeptide. Native ligands or ligand mimics that have a cysteine residue that forms a disulfide loop or a tyrosine residue that can bind the alpha1 domain alpha helix of a MHC class II molecule. In particular, the interaction between the mimetic compound occurs at residues alanine 61, leucine 60, and/or glutamine 57 of the alpha1 domain alpha helix of a MHC class II molecule, thereby inhibiting the activity of the bacterial toxin.

The steps for designing a mimetic from a compound having a specific target property comprises first ascertaining the critical components of the compound. This may be accomplished by substituting amino acid residues of a peptide for example. Those parts or residues that have been identified'as the active region of the compound are defined as its “pharmacophore.” The mimetic structure can then be designed using the physical properties of the compound. A template molecule containing functional groups, which mimic the pharmacophore, may be used to synthesize the mimetic. In the present invention, the template molecule is the 12-mer peptide 6343, which demonstrated that the N-terminal end, in particular the CMY, interact with the MHC class II molecule.

Compositions-Peptides

In one embodiment of this invention compositions comprising isolated and purified peptides, derivatives, and/or mimetics having amino acid sequences related to a conserved region of the staphylococcal enterotoxins and streptococcal pyrogenic exotoxins are provided. These peptides may be used for directly inhibiting toxic activity of bacterial toxins or for eliciting an immunogenic response in mammals, including responses which provide protection against, or reduce the severity of, toxic shock from bacterial toxins, such as but not limited to staphylococcal or streptococcal pyrogenic exotoxins. Preferably, these bacterial toxins are staphylococcal or streptococcal pyrogenic exotoxins and more preferably SEB.

The peptides, derivatives, or mimetics thereof of this invention may be prepared by synthetic methods or by recombinant DNA methods well known in the art. Peptides of the present invention and antibodies directed against these peptides relate to a conserved region of several bacterial toxins, preferably bacterial superantigens. “Bacterial superantigens”, as defined herein, are toxins, primarily from gram-positive bacteria, which strongly stimulate large populations of T cells. Superantigens first bind the major histocompatibility complex II (MHCII) as a binary complex, then bind T cell antigen receptors (TCR) in a Vβ-specific manner (Fleischer and Schrezenmeier, B. H., J. Exp. Med. 167:1697-1707, 1988; Mollick et al., Science 244:817-820, 1989; Janeway et al, Immunol. Rev. 107:61-88, 1989; White et al., Cell 56:27-35, 1989). Bacterial toxins comprise of two major toxin groups: endotoxins and exotoxins. Exotoxins further comprise enterotoxins, where superantigens include Staphylococcal enterotoxins and streptococcal pyrogenic exotoxins.

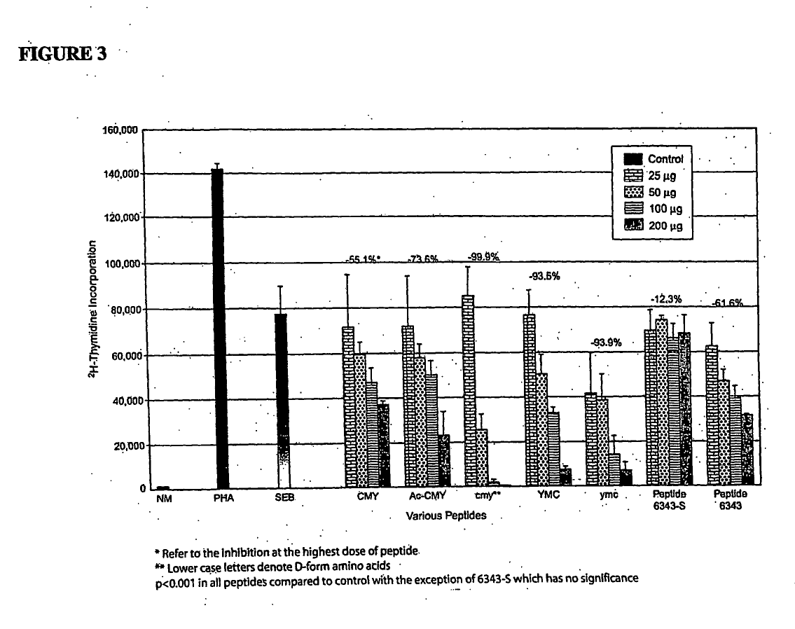

The peptides of this invention can be subject to various modifications that provide for certain advantages in their use. For example, amino acids in the D-conformation are preferably substituted for those amino acids in the natural L-conformation in order to increase in vivo stability of the peptides, while still retaining biological activity (Senderoff et al., J. Pharm. Sci. 87:183-189, 1998). Specifically, the D-conformation of amino acids for CMY and YMC are most preferred. They have been demonstrated to significantly inhibit blastogenesis compared to the peptide of 12 amino acids or 12-mer (see FIG. 3). Additionally, since the D-conformations of the trimer may be as or more effective than the L-conformations of the trimer, the trimers having D-conformation may not be as easily degraded by various enzymes in the gastrointestinal system, i.e., gut or circulation. Thus, these D-conformation trimers may result in a longer half-life in plasma and are preferable in treating subjects, i.e., humans.

In addition, retro-inverso peptides which contain NH—CO bonds instead of CO—NH peptide bonds have been shown to be more resistant to proteolysis than L-conformation peptides and yet mimic natural L-conformation peptides with respect to poly- and monoclonal antibody binding (Chorev and Goodman, M. Trends Biotechnol. 3:438-445, 1995). Thus, those peptides having at least one amino acid in the D-conformation, preferably at the amino terminal of the molecule and which retain functional activity are also considered part of the invention, as well as retro-inverso peptides containing one or more of the amino acid sequences of the invention and which retain functional activity.

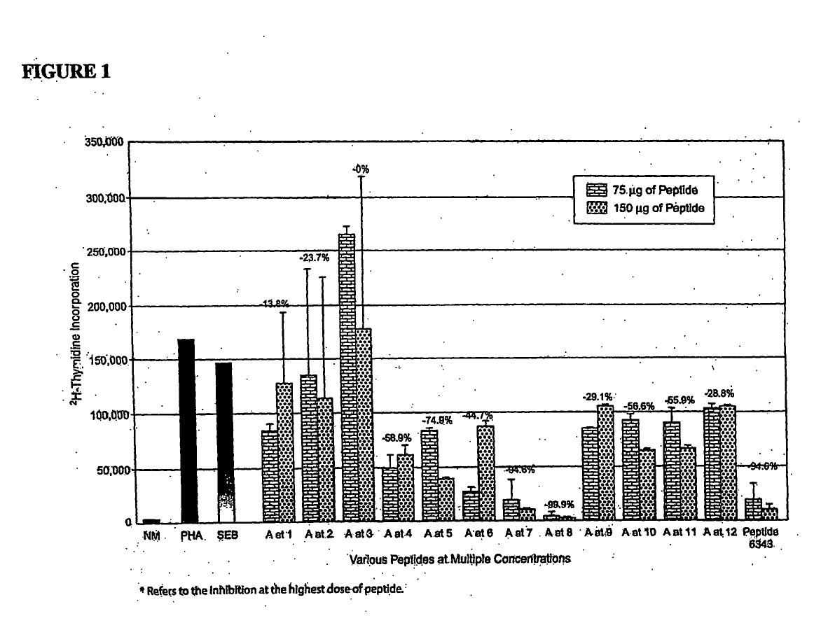

Both the D- and L-conformations of the trimers having amino acid substitutions as described in FIGS. 1, 3-5 suggest that amino acid side chains and their interactions may be an important site for blocking superantigen activity.

Also contemplated in this invention are any peptides, derivatives, or mimetics designed to block key hydrophobic interactions with the MHC class II molecule, or T cell receptor. The tyrosine residue side chains of the peptide, derivative, or mimetic that interact hydrophobically with residues of the MHC class II molecule are of particular interest Peptides comprising the amino acid sequence TEHEGN (SEQ ID NO: 7), and more particularly, those peptides that have glutamic acid, threonine, and glycine are also important for their hydrophobic interactions with the MHC class II molecule.

The preferred peptides of the invention are those which exclude full length native toxin molecules. The preferred peptides of this invention are not toxic, but toxic peptides may be useful in this invention, for example, in eliciting antibodies in a non-human system. The most preferred peptides of the invention do not contain amino acid sequences in the sequence in which they are found in any particular native toxin molecule.

The instant invention also encompasses homogeneous or heterogeneous polymers of the peptides disclosed herein (e.g., concatenated, cross-linked and/or fused identical peptide units or concatenated, cross-linked and/or fused diverse peptide units), and mixtures of the peptides, polymers, and/or conjugates thereof. The amino acid cysteine “C” is used to facilitate cross-linking through the formation of disulfide bonds. The amino acid glycine “G” or serine “S” may be used as a spacer residues.

Low molecular weight species of the invention are useful themselves in inhibiting superantigen induced T cell proliferation and/or reducing, inhibiting, or eliminating the deleterious effects of bacterial toxins, in particular exotoxins in vivo, either when used alone or in combination with another form of therapy, for example, antibodies directed against cytokines.

Linkers useful in this invention may, for example, be simply peptide bonds, or may comprise amino acids, including amino acids capable of forming disulfide bonds, but may also comprise other molecules such as, for example, polysaccharides or fragments thereof. The linkers for use with this invention may be chosen so as to contribute their own immunogenic effect which may be either the same, or different, than that elicited by the consensus sequences of the invention. For example, such linkers may be bacterial antigens which also elicit the production of antibodies to infectious bacteria. In such instances, for example, the linker may be a protein or protein fragment of an infectious bacteria, or a bacterial polysaccharide or polysaccharide fragment.

Compositions-Nucleic Acids

This invention further relates to isolated and purified nucleic acid molecules which encode the peptides, derivatives, or mimetics of the invention as previously described. The encoded peptides may be monomers, polymers, or they may be linked to other peptide sequences (i.e., they may be fusion proteins). Other features of the invention include vectors which comprise the nucleic acid molecules of the invention operably linked to promoters, as well as transformed cell lines, such as prokaryotic (e.g., E. coli) and eukaryotic (e.g., CHO and COS) cells possessing the nucleic acid molecules of the invention. Vectors and compositions for enabling production of the peptides in vivo, i.e., in the individual to be treated or immunized, are also within the scope of this invention.

The nucleic acids encoding the peptides of the invention can be introduced into a vector, such as a plasmid, cosmid, phage, virus or mini-chromosome, and inserted into a host cell or organism by methods well known in the art. See, for example, Sambrook et al., (Molecular Cloning: A Laboratory Manual, Cold Spring Harbor Laboratory, Cold Spring Harbor, N.Y., 1989), which is incorporated herein by reference. In general, the vectors containing these nucleic acids can be utilized in any cell, either eukaryotic or prokaryotic, including mammalian cells (e.g., human (e.g., HeLa), monkey (e.g., COS), rabbit (e.g., rabbit reticulocytes), rat, hamster (e.g., CHO and baby hamster kidney cells) or mouse cells (e.g., L cells), plant cells, yeast cells, insect cells or bacterial cells (e.g., E. coli). The vectors which can be utilized to clone and/or express these nucleic acids are the vectors which are capable of replicating and/or expressing the nucleic acids of the invention in the host cell in which the nucleic acids are desired to be replicated and/or expressed. See, e.g., F. Ausubel, et al., Current Protocols in Molecular Biology, Greene Publishing Associates and Wiley-Interscience (1992) and Sambrook, et al. (1989) for examples of appropriate vectors for various types of host cells. Strong promoters compatible with the host into which the gene is inserted may also be used. These promoters may be inducible. The host cells containing the nucleic acids can be used to express large amounts of the protein useful for producing pharmaceuticals, diagnostic reagents, vaccines, and therapeutics.

The nucleic acids can also be used, for example, in the production of peptides for diagnostic reagents, vaccines, and therapies for pyrogenic exotoxin and endotoxin related diseases. For example, vectors expressing high levels of peptide can be used in immunotherapy and immunoprophylaxis, after expression in humans. Such vectors include retroviral vectors and also include direct injection of DNA into muscle cells or other receptive cells, resulting in the efficient expression of the peptide, using the technology described, for example, in Wolff et al., (Science 247:1465-1468, 1990, Wolff et al., Human Molecular Genetics 1:363-369, 1992) and Ulmer et al., (Science 259:1745-1749, 1993). See also, for example, WO 96/36366 and WO 98/34640.

Compositions-Antibodies

In another embodiment of this invention, antibodies are provided which react with peptides, derivatives, or mimetics of the invention. The term “antibodies” is used herein to refer to immunoglobulin molecules and immunologically active portions of immunoglobulin molecules. Exemplary antibody molecules are intact immunoglobulin molecules, substantially intact immunoglobulin molecules and portions of an immunoglobulin molecule, including those portions known in the art as Fab, Fab′, F(ab′)₂ and F(v) as well as chimeric antibody molecules.

An antibody useful in the present invention is typically produced by immunizing a mammal with one or more peptides, derivatives, or mimetics thereof of the invention, or a structurally and/or antigenically related molecule, to induce, in the mammal, antibody molecules having immunospecificity for immunizing peptide or peptides. The peptide(s), derivative(s), or mimetic(s) thereof or related molecule(s) may be monomeric, polymeric, conjugated to a carrier, and/or administered in the presence of an adjuvant. In one embodiment, the peptides of the invention are linked to spacers, such as but not limited to amino acids glycine or serine, and conjugated to adjuvants, including tetanus toxoid.

Another embodiment of this invention is directed peptides as haptens conjugated to a larger carrier molecule, such as, for example, a protein. As with other peptides, the molecular weight of the peptide alone, or when conjugated to a carrier, or in the presence of an adjuvant, is related to its immunogenicity. Commonly used carriers that are chemically coupled to peptides include, but are not limited to, bovine serum albumin (BSA), keyhole limpet hemocyanin (KLH), and thyroglobulin (Eds: Ed Marlow, David Lane. Antibodies. 1988. Cold Spring Harbor Press, Chapter 5, p. 78). Thus, the peptide may vary in molecular weight in order to enhance its antigenicity or immunogenicity. The total size of the peptide is only limited to its ability to be physiologically tolerated. A further embodiment of this invention relates to peptides conjugated to hexanoic acid, which has been reported to induce or elicit antibodies to the attached peptides. The antibody molecules elicited by the peptides of the invention, may then be collected from the mamrnmal if they are to be used in immunoassays or for providing passive immunity.

For the production of antibodies, various hosts, including goats, rabbits, sheep, rats, mice, humans, and others, may be immunized by injection with one or more of the peptides of the invention, or any immunogenic and/or epitope-containing fragment or oligopeptide thereof, which have immunogenic properties. Depending on the host species, various adjuvants may be used to increase the immunological response. Non-limiting examples of suitable adjuvants include Freund's (incomplete), mineral gels such as aluminum hydroxide or silica, and surface active substances such as lysolecithin, pluronic polyols, polyanions, peptides, oil emulsions, KLH, and dinitrophenol. Adjuvants typically used in humans include BCG (bacilli Calmette Guérin) and Corynebacterium parvumn.

The antibody molecules of the present invention may be polyclonal or monoclonal. Monoclonal antibodies may be produced by methods well known in the art Monoclonal antibodies may be used to test for the presence of specific antigens, to study cross-reactivity among antigens, and to purify antigens. A monoclonal antibody is specific for a certain epitope, occurring only on certain proteins. The hybridoma technique described originally by Kohler and Milstein (Eur. J. Immunol. (1976) 6:511) is widely applied to produce hybrid cell lines that secrete high levels of monoclonal antibodies against many specific antigens (see, for example, Example 10). The hybrid cell is screened on the basis of the ability to grow on a specific medium in which neither the pure spleen cell, nor the pure myeloma cell can grow. The hybrid cell possesses the property of the immortal character of the tumor cell and the specific antibody production since it has a double complement of genes. The hybridoma and its clones may be injected into animals to induce antibody-secreting myelomas, or they may be grown in mass culture to produce a specific antibody. A single hybridoma cell clone produces large amounts of identical antibody against a single epitope (antigenic determinant). Fragments of immunoglobulin molecules may also be produced and used by methods commonly known in the art. For example, Fab fragments which maintain the ability to bind specific antigens is within the scope of this invention. In addition, human antibodies may be produced in transgene animals which express a “human” immune system or antibodies raised in species other than humans may be humanized according to methods known in the art. See, for example, U.S. Pat. No. 6,180,370 to Queen, et al.

The antibodies of this invention may further be contained in various carriers or media, including blood, plasma, serum (e.g., fractionated or unfractionated serum), hybridoma supernatants and the like. Alternatively, the antibody of the present invention is isolated to the extent desired by well known techniques such as, for example, by using DEAE Sephadex, or affinity chromatography. The antibodies may be purified so as to obtain specific classes or subclasses of antibody such as IgM, IgG, IgA, IgG₁, IgG₂, IgG₃, IgG₄ and the like. Antibodies of the IgG class are preferred for purposes of passive protection.

Methods of Use-Peptides

Although the mechanism of action of the peptides of this invention is unclear, these peptides, derivatives, or mimetics thereof, are believed to be used to provide active immunization for the prevention of disease related to the detrimental effects of bacterial toxins, specifically, staphylococcal and streptococcal pyrogenic exotoxinsl Antibodies produced according to this invention may also be used to provide passive immunization therapy.

The peptides, derivatives, or mimetics of this invention appear to preferably inhibit the toxic activity of bacterial toxins, including endotoxins and exotoxins via a mechanism independent of the generation of antibodies. Accordingly, the peptides, derivatives, or mimetics of this invention are used for preventing or treating symptoms due to release of bacterial exotoxins and endotoxins, either through their direct action or by their ability to elicit the generation of protective antibodies.

In another embodiment of the invention, the peptides, derivatives, or mimetics of this invention may induce antibodies which react with a variety of bacterial toxins, including staphylococcal and streptococcal pyrogenic exotoxins (preferably with at least two, more preferably with at least four, and most preferably with at least seven of the pyrogenic exotoxins, e.g. A, B, C, E, F, G, K, M). These peptides are also useful in inducing antibodies for therapies for preventing and/or treating toxic shock syndrome, septic shock, food poisoning, and/or any other bacterial toxin-related disease or condition.

A further embodiment of this invention relates to the peptides, derivatives, and mimetics of this invention which are also useful in diagnostic assays and kits to detect the presence of antibodies to staphylococcal and streptococcal pyrogenic toxins, preferably exotoxins, and to aid in the diagnosis of diseases related to the presence of these toxins.

The peptides, derivatives, or mimetics of this invention may further be useful for protecting against, or ameliorating the effects of autoimmune diseases which are associated with, or are the result of, the presence of bacterial toxins.

Methods of Use-Antibodies

The antibodies provided by this invention react with peptides, derivatives, or mimetics thereof of the invention, in addition to a variety of bacterial toxins such as staphylococcal and streptococcal pyrogenic exotoxins. These antibodies are believed to be useful for passive immunization therapy to increase resistance to or prevent toxic shock syndrome or septic shock, or other disease related to the presence of bacterial toxins. The antibodies can also be useful in protecting against or ameliorating the effects of autoimmune diseases which are associated with, or are the result of, the presence of bacterial toxins. The antibodies of the invention will also be useful in diagnostic tests and kits for detecting the presence of bacterial toxins such as staphylococcal and streptococcal pyrogenic exotoxins and/or endotoxins.

In another embodiment, the antibodies of this invention have a number of diagnostic and therapeutic uses. The antibodies can be used as an in vitro diagnostic agent to test for the presence of various bacterial toxins in biological samples in standard immunoassay protocols and to aid in the diagnosis of various diseases related to the presence of bacterial toxins. Preferably, the assays which use the antibodies to detect the presence of bacterial toxins in a sample involve contacting the sample with at least one of the antibodies under conditions which will allow the formation of an immunological complex between the antibody and the toxin that may be present in the sample. The formation of an immunological complex if any, indicating the presence of the toxin in the sample, is then detected and measured by suitable means. Such assays include, but are not limited to, radioimmunoassays, (RIA), ELISA, indirect immunofluorescence assay, Western blot and the like. The antibodies may be labeled or unlabeled depending on the type of assay used. Labels which may be coupled to the antibodies include those known in the art, such as, but not limited to, enzymes, radionucleotides, fluorogenic and chromogenic substrates, cofactors, biotin/avidin, colloidal gold and magnetic particles. Modification of the antibodies allows for coupling by any known means to carrier proteins or peptides or to known supports, for example, polystyrene or polyvinyl microliter plates, glass tubes or glass beads and chromatographic supports, such as paper, cellulose and cellulose derivatives, and silica. Preferably, a high throughput method of screening for bacterial toxins may be used with a microchip, glass slide, or other similar support.

Such assays may be, for example, of direct format (where the labeled first antibody reacts with the antigen), an indirect format (where a labeled second antibody reacts with the first antibody), a competitive format (such as the addition of a labeled antigen), or a sandwich format (where both labeled and unlabeled antibody are utilized), as well as other formats described in the art. In one such assay, the biological sample is contacted to antibodies of the present invention and a labeled second antibody is used to detect the presence of bacterial toxins, to which the antibodies are bound.

The antibodies of the present invention are also useful as therapeutic agents in the prevention and treatment of diseases caused by the deleterious effects of bacterial toxins. Antibodies for eliciting passive immunity in mammals, preferably humans, are preferably obtained from other humans previously inoculated with compositions comprising one or more of the consensus amino acid sequences of the invention. Alternatively, antibodies derived from other species may also be used. Such antibodies used in therapeutics suffer from several drawbacks such as a limited half-life and a propensity to elicit a deleterious immune response. Several methods have been proposed to overcome these drawbacks. Antibodies made by these methods are encompassed by the present invention and are included herein. One such method is the “humanizing” of non-human antibodies by cloning the gene segment encoding the antigen binding region of the antibody to the human gene segments encoding the remainder of the antibody. Only the binding region of the antibody is thus recognized as foreign and is much less likely to cause an immune response. Queen, et al. describe such antibodies (Proc. Natl. Acad. Sci. USA 86(24):10029, 1989), incorporated herein by reference.

Pharmaceutical Compositions

This invention relates to compositions and methods for providing protection against, or reducing the severity of, toxic shock syndrome, septic shock, food poisoning, and autoinunune diseases which are associated with toxin producing bacteria. Further, this invention relates to methods of preventing or inhibiting the previously mentioned diseases, syndromes, and conditions in mammals by directly administering peptides, derivatives, or mimetics to the rammal, preferably human, in an effective amount.

The pharmaceutical compositions of this invention contain a pharmaceutically and/or therapeutically effective amount of at least one peptide with or without a covalently bound carrier thereof, antibody, or nucleic acid encoding a peptide of this invention. In one embodiment of the invention, the effective amount of peptide per unit dose is an amount sufficient to inhibit T cell proliferation stimulated by bacterial toxins, specifically staphylococcal and/or streptococcal pyrogenic exotoxins. In another embodiment of this invention, the effective amount of peptide per unit dose is an amount sufficient to prevent, treat, or protect against the toxic effects of bacterial toxins, including but not limited to, diarrhea, fever, chills, vomiting, sore throat, headache, sepsis, and heart failure. Any reduction, amelioration, or elimination of one or more of these symptoms caused by bacterial toxins is understood to be a useful dose. Furthermore, the amount of peptide per unit dose depends, among other things, on the species of mammal inoculated, the body weight of the mammal, and the chosen inoculation regiment, all of which are assessed by one skilled and knowledgable in the art.

At least three different classes of pharmaceutical compositions are provided by this invention. One being pharmaceutical compositions comprising peptides, derivatives, or mimetics thereof, which act directly to inhibit toxicity of bacterial toxins. In another pharmaceutical composition, the peptides are provided in an amount suitable to elicit an immunogenic response themselves. Another embodiment encompasses pharmaceutical compositions comprising antibodies generated in response to the peptides of this invention. Such pharmaceutical compositions are useful for providing passive protection against bacterial toxins.

Modes of Administration

The peptides, derivatives, mimetics, and/or antibodies of the invention are intended to be provided to the recipient subject in an amount sufficient to prevent, or attenuate the severity, extent, or duration of the deleterious effects of bacterial toxins. Such deleterious effects may be manifested as toxic shock syndrome, septic shock, food poisoning, and autoimmune diseases associated with toxin producing bacteria. Non-limiting examples of symptoms associated with bacterial toxins which may be prevented or treated in accordance with this invention include, but are not limited to, fever, chills, vomiting, sore throat, headache, diarrhea, decreased urine output, severe myalgias, vaginal, oropharyngeal, or conjunctival hyperemia, disorientation or alteration in consciousness, desquamation (typically palms and soles), lowering of blood pressure (shock), kidney failure, liver failure, and heart failure.

Peptides

The peptides, derivatives, or mimetics thereof, of this invention comprising low molecular weight species are useful for inhibiting peripheral blood mononuclear cell (PBMC) proliferation and/or for reducing, inhibiting, or eliminating the deleterious effects of bacterial exotoxins in vivo, either when used alone or in combination with other types of therapy, for example passive immunization. Further, these peptides are administered via routes that include, but are not limited to intravenous, intramuscular, subcutaneous, intradermal, intraperitoneal, intrathecal, intraplerural, topical, and the like. Intravenous administration may be the preferred route of administration in a mammal having acute symptoms related to diseases associated with toxin producing bacteria.

Administration may also be by transmucosal or transdermal means. For transrnucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be by, for example, nasal sprays or suppositories. For oral administration, the peptides of this invention, or variants thereof are formulated into conventional oral administration forms such as capsules, tablets, and toxics.

A system for sustained delivery of the peptides of the invention may also be used. For example, a delivery system based on containing a peptide in a polymer matrix of biodegradable microspheres may be used (Jeong et al., Nature 388:860-862, 1997). One such polymer matrix includes the polymer poly(lactide-co-glycolide) (PLG). PLG is biocompatble and can be introduced intravenously or orally. Following injection of the microspheres into the body, the encapsulated protein is released by a complex process involving hydration of the particles and drug disolution. The duration of the release is mainly governed by the type of PLG polymer used and the release of modifying excipients (Bartus, et al., Science 281:1161-1162, 1998).

Generally, it is desirable to provide the recipient with a dosage of peptide of at least about 150 mgs/ kg body weight, preferably at least about 100 mgs/ kg body weight, and more preferably at least about 50 mgs/ kg body weight or greater of the recipient. A range from about 50 mgs/ kg body weight to about 100 mgs/ kg body weight is preferred, although a lower or higher dose may be administered. Without being bound by theory,.the dose is believed to be effective to block the toxin pathway, which in turn is capable of preventing or inhibiting the onset of poisoning by bacterial toxin and in particular, lethal shock induced by the combination of the LPS and one or more of the superantigens in the recipient, preferably human.

Peptides as Inmunogens

The term “unit dose” as it pertains to the use of the peptides of this invention to induce an immune response refers to physically discrete units suitable as unitary dosages for mammals, each unit containing a predetermined quantity of active material (peptide) calculated to produce the desired immunogenic effect in association with the required diluent or excipient.

When the peptide of the invention is used as an immunogen, the pharmaceutical composition contains an effective, immunogenic, amount of peptide of the invention. The peptide may be mixed with an adjuvant. The peptide also may be bound to a non-toxic non-host protein carrier to form a conjugate or it may be bound to a saccharide carrier and/or a non-toxic non-host protein carrier to form a conjugate. The effective amount of peptide per unit dose sufficient to induce an immune response depends on, among other things, the species of mammal inoculated, the body weight of the mammal, and the chosen inoculation regimen, as well as the presence or absence of an adjuvant These conditions are commonly known in the art such that the skilled artisan would be able to properly dose the patient.

Inocula are typically prepared as a solution in a physiologically acceptable diluent or excipient such as saline, phosphate-buffered saline and the like to form an aqueous pharmaceutical composition. When used as an immunogen, the peptide can be mixed with an adjuvant. Any pharmaceutically acceptable adjuvant is suitable for use with the peptides of this invention, for example, aluminum, and stearyl tyrosine. The peptide also can be bound to a non-toxic, non-host protein carrier to form a conjugate or it may be bound to a saccharide carrier to form a conjugate. Various methods for conjugating peptides are known in the art. See, for example, W. E. Dick and M. B. Beurret (Cruse J M, Lewis R E Jr (eds): “Conjugate Vaccines”. Contrib. Microbiol. Immunol. Basel, Karger, 1989, vol. 10, pp. 48-114).

Inocula typically contain peptide concentrations of about 100 micrograms to about 5 milligrams per inoculation or unit (dose), preferably about 3 micrograms to about 500 micrograms per dose, most preferably about 100 micrograms to 250 micrograms. When used as an immunogen, inocula for a human or similarly sized mammal typically contain peptide concentrations of about 1 to 5 micrograms/kg body weight of the mammal per inoculation dose. The use of higher or lower amounts are contemplated. The nmiber of doses is preferably 3, but any fewer or more are contemplated. Standard procedures to determine dose response relationships known to those skilled in the art may be used to determine optimum doses of peptide to be used either to prevent or treat toxic or septic shock or other related diseases or conditions, or to raise antibodies for the prevention or treatment thereof.

The route of inoculation of the peptides of the invention is typically parenteral and is preferably intravenous, intramuscular, sub-cutaneous, and the like, which can result in eliciting antibodies protective against the deleterious effects of staphylococcal and streptococcal pyrogenic exotoxins. The dose being administered at least once. In order to increase the antibody level, at least one booster dose may be administered after the initial injection, preferably at about 4 to 6 weeks after the first dose. Subsequent doses may be administered as needed.

Antibodies

Antibodies of this invention are generally adminstered with a pharmaceutically and physiologically acceptable diluent, excipient, or vehicle therefore. A physiologically acceptable diluent or excipient is one that does not cause an adverse physical reaction upon administration and one in which the antibodies are sufficiently soluble and retain their activity to deliver a therapeutically effective amount of the compound. The therapeutically effective amount and method of administration of the antibodies may vary based on the individual patient, the indication being treated and other criteria evident to one of ordinary skill in the art. A therapeutically effective amount of the antibodies is one sufficient to attenuate the dysfunction without causing significant side effects such as non-specific T cell lysis or organ damage.

Routes of administration of the antibodies include, but are not limited to, parenteral, and direct injection into an affected site. Parenteral routes of administration include but are not limited to intravenous, intramuscular, intraperitoneal and subcutaneous.

This invention includes compositions of the antibodies described above, suitable for parenteral administration including, but not limited to, pharmaceutically acceptable sterile, isotonic solutions. Such solutions include, but are not limited to, saline and phosphate buffered saline for intravenous, intramuscular, intraperitoneal, subcutaneous or direct injection into a joint or other area.

Upon providing the antibodies of the present invention to a recipient mammal, preferably a human, the dosage of administered antibodies varies depending on factors such as the mammal's age, weight, height, sex, general medical condition, previous medical history, and the like.

In general, it is desirable to provide the recipient with a dosage of antibodies ranging from about 5 mg/kg to about 20 mg/kg body weight of the mammal, although a lower or a higher dose may be administered. In general, the antibodies will be administered intravenously or intramuscularly. Intravenous immunoglobulin (IVIG) can generally be given with a loading dose of 200 mg/lkg, with monthly injections of about 100 mg/kg. High dose IVIG may be given at 400-800 mg/kg, for antibody deficient patients. See, for example, The Merck Manual of Diagnosis and Therapy, 16th Edition, (Berkow R and Fletcher A J, Eds.), Merck Research Laboratories, Rahway, N.J. (1992).

When a composition of the invention is used to induce an immunogenic response, specifically inducing antibodies, at least one booster dose may be administered after the initial injection, preferably at about 4 to 6 weeks after the first dose, in order to increase the antibody level. Subsequently, doses may be administered as appropriate.

To monitor the antibody response of individuals administered the compositions of the invention, antibody titers may be determined. In most instances it will be sufficient to assess the antibody titer in serum or plasma obtained from such an individual. Decisions as to whether to administer booster inoculations or to change the amount of the composition administered to the individual may be at least partially based on the titer.

The titer may be based on either an immunobinding assay which measures the concentration of antibodies in the serum which bind to a specific antigen, i.e. peptide or toxin; or bactericidal assays which measure the ability of the antibodies to participate with complement in killing bacteria. The ability to neutralize in vitro and in vivo biological effects of the pyrogenic exotoxins may also be assessed to determine the effectiveness of the treatment.

The presence of the antibodies of the present invention, either polyclonal or monoclonal, can be determined by various assays. Assay techniques include, but are not limited to, immunobinding, immunofluorescence (IF), indirect immunofluorescence, inununoprecipitation, ELISA, agglutination and Western blot techniques.

General Prophylactic and/or Therapeutic Uses

The administration of the agent compositions of this invention, including peptides, derivatives, mimetics, and antibodies, may be for either “prophylactic” or “therapeutic” purpose. When provided prophylactically, the agents are provided in advance of any symptom. The prophylactic administration of the agent serves to prevent or ameliorate any subsequent deleterious effects of, toxic shock syndrome, septic shock, food poisoning, and autoimmune diseases which are associated with toxin producing bacteria. When provided therapeutically, the agent is provided at (or shortly after) the onset of a symptom of infection with bacteria, preferably expressing staphylococcal or streptococcal pyrogenic exotoxins. The agent of the present invention may, thus, be provided either prior to the anticipated exposure to bacteria toxins (so as to attenuate the anticipated severity, duration or extent of disease symptoms) or after the initiation of the infection. The agent may also be provided to individuals of high risk for bacterial infection and subsequence toxic responses, particularly with bacteria expressing staphylococcal or streptococcal pyrogenic exotoxins.

Also contemplated are therapies based upon vectors, such as viral vectors containing nucleic acid sequences coding for the peptides described herein. These molecules, developed so that they do not provoke a pathological effect, will stimulate the immune system to respond to the peptides.

For all therapeutic, prophylactic, and diagnostic uses, the peptides of the invention, alone or linked to a carrier, as well as antibodies and other necessary reagents and appropriate devices and accessories may be provided in kit form so as to be readily available and easily used.

Where immunoassays are involved, such kits may contain a solid support, such as a membrane (e.g., nitrocellulose), a bead, sphere, test tube, rod, and so forth, to which a receptor such as an antibody specific for the target molecule will bind. Such kits can also include a second receptor, such as a labeled antibody. These kits may be used for sandwich assays to detect toxins. Kits for competitive assays are also envisioned.

Any of the therapeutic methods described above may be applied to any individual in need of such therapy, including, for example, mammals such as dogs, cats, cows, horses, rabbits, monkeys, and most preferably, humans.

The following examples illustrate certain embodiments of the present invention, but should not be construed as limiting its scope in any way. Certain modifications and variations will be apparent to those skilled in the art from the teachings of the foregoing disclosure and the following examples, and these are intended to be encompassed by the spirit and scope of the invention.

Unless defined otherwise, all technical and scientific terms used herein have the same meanings as commonly understood by one of skill in the art to which the disclosed invention belongs. Although any methods and materials similar or equivalent to those described herein may be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to “the antibody” is a reference to one or more antibodies and equivalents thereof known to those skilled in the art, and so forth.

The present invention will now be described by way of examples, which are meant to illustrate, but not limit, the scope of the invention.

### EXAMPLES

### Example 1

### Materials and Methods Superantigen

All superantigens were purchased from Toxin Technology (Sarasota, Fla.).

Peptide Construction

A number of peptides were constructed based on the consensus sequences of SE/SPE toxins. Peptides were constructed and purified by HPLC according to standard methods (Merrifield, B., Science 232:341-347, 1986; Patarroyo, et al., Nature 328:629-632, 1987). HPLC analysis was performed and revealed that all peptides had a purity of greater than 95%. Peptides were constructed by Multiple Peptide Systems (San Diego, Calif.) or in the protein/DNA Technology Center at Rockefeller University (New York, N.Y.).

Blastogenesis/Proliferation Assays

Human peripheral blood mononuclear cells (PBMCs) were isolated by standard Ficoll-Hypague techniques and adjusted to 2×10⁶ cells/ml. PBMC (2×10⁵) in 200 microliters of complete medium (RPMI+10% human AB senun) were placed in 96 well titer plates and stimulated with varying doses of superantigen or a combination of each toxin with varying doses of peptides. The cells were incubated for 6 days and the results were measured by tritiated thymidine incorporation. CPM represents counts per minute. The data presented are the results of the average of 3 different experiments. All tests were performed in triplicate.

Viability Studies

Human PBMCs were isolated as described above and 2×10⁵ cells were placed in 96 well titer plates. PHA was added at a concentration of 5 micrograms/well. All peptides were added to PHA at a concentration of 200 micrograms/well. The plates were incubated at 37° C. in a CO2 incubator for 72 hours at which time 3 microCuries of tritiated thymidine was added to the cells. After a further incubation of 18 hours, the cells were harvested and the CPM of tritiated thymidine was counted. All experiments were carried out in triplicate.

A second viability test was performed by plating 2×10⁵ PBMCs in 96 well titer plates. Various concentrations of peptide were added. An aliquot of cells with and without peptide was stained with Trypan Blue each day for five days to observe viability of the cells in the presence or absence of peptide.

Animal Toxic Shock Experiments

Eight weeks old female BALB/c mice were used for all experiments. Animals were house at the Rockefeller University Laboratory Animal Research Facility (LARC) and experiments were undertaken as described herein. All mice were sensitized with 0.001 mg lipopolysacclaride (LPS) and 20 mg of D-Galactosarnine via intraperitoneal injection (Blank, et al. Eur. J. Immunol. 27(4):825-833). Eight hours later, mice were injected with varying doses of superantigen that had been shown to cause 100% lethality. In protection experiments, two hours prior to superantigen injection, saline or 1.5 mg of the peptide was administered to the experimental mice by subcutaneous injection. One hour prior to superantigen injection, the mice were injected again with either saline or 1.5 mg peptide (3.0 mg total). One hour after the second injection, all of the mice were challenged with the appropriate dose of toxin, i.e., superantigen, (via intraperitoneal injection) and the mice were observed for 24-48 hours.

Antibody Production

Three WNZ female rabbits (3 kg) are used for the injections. The initial injection is 500 micrograms of polymerized peptide in complete Freud's Adjuvant. Two boosters of 250 micrograms per rabbit in incomplete adjuvant are then separately administered 21 days apart and 21 days after the initial injection. Antibody titers of 1×10⁶ml are routinely obtained using ELISA plates coated with a peptide of this invention. The larger polymerized peptides are known to be more immunogenic (Patarroyo, et al., Nature 332:158-161, 1988).

The IgG fraction of the serum antibodies directed against a peptide of the invention are isolated using a protein A column for further enrichment. These antibodies, in addition to those raised against other bacterial toxin regions, are used to demonstrate that the peptide anti-serum is able to recognize the conserved regins of various bacterial toxins, but not that of TSST-1. Further, the antibodies show strong inhibition of blastogenesis to all of the bacterial toxins tested. In addition, nanogram amounts of total IgG is determined to be sufficient to achieve 93-100% superantigen inhibition. However, a high titered antibody directed against enriched group A streptococcal carbohydrate is unable to block the biological properties of the toxins.

### Example 2

### Alanine Substitution Constructs

In order to assess the contribution of specic amino acids in peptide sequences related to the consensus sequence of SEB, where the 12-mer peptide 6343 (CMYGGVTEHEGN; SEQ ID NO: 1) has previously been reported to induce toxin inhibition, various peptides were constructed by substituting a single amino acid alanine for each amino acid of the 12-mer peptide 6343 leaving all other amino acids of the peptide intact. Alanine was chosen as it is a relatively neutral peptide single amino acid substitution. Examples of the constructs are listed in Table 1, where the substituting alanine (A) is in bold-faced type and underlined.

TABLE 1
Alanine Substitution Constructs

| | |
|---|---|
| AMYGGVTEHEGN | CMYGGVAEHEGN |
| (SEQ ID NO:8) | (SEQ ID NO:14) |
| CAYGGVTEHEGN | CMYGGVTAHEGN |
| (SEQ ID NO:9) | (SEQ ID NO:15) |
| CMAGGVTEHEGN | CMYGGVTEAEGN |
| (SEQ ID NO:10) | (SEQ ID NO:16) |
| CMYAGVTEHEGN | CMYGGVTEHAGN |
| (SEQ ID NO:11) | (SEQ ID NO:17) |
| CMYGAVTEHEGN | CMYGGVTEHEAN |
| (SEQ ID NO:12) | (SEQ ID NO:18) |
| MYGGATEHEGN | CMYGGVTEHEGA |
| (SEQ ID NO:13) | (SEQ ID NO:19) |

As shown in FIG. 1 and Table 1, the substitution of alanine for any of the first three amino acids at the N-terminus, .i.e. Cysteine, C (SEQ ID NO: 8), Methionine, M (SEQ ID NO: 9), and Tyrosine, Y (SEQ ID NO: 10), resulted in significant loss of the inhibitory properties of the peptide. Peptides with alanine substitution after the third amino acid resulted in relatively little loss of activity. Alanine substitution at amino acid residues 4-8 inhibited biological activity. Substitution of any of the first three amino acids resulted in a much greater loss of inhibition. Alanine substitution of tyrosine resulted in a complete loss of the inhibitory properties of the 12-mer peptide, indicating that tyrosine is most crucial for inhibitory activity of the peptide. Further, FIG. 1 surprisingly shows that alanine substitution of the seventh and eighth amino acids (A at 7; A at 8) from the N-terminal resulted in better inhibition of biological activity than the original 12-mer peptide (6343) itself.

### Example 3

### Amino Acid Removal Studies

A series of peptides were prepared in which a single amino acid was removed in succession from the N-tenninal end or from the C-terminal end of the peptide. The constructs were designed starting with the original 12-mer peptide (6343) as described in Table 2.

TABLE 2

| | |
|---|---|
| N-Terminal Deletions | C-Terminal Deletions |

| | |
|---|---|
| MYGGVTEHEGN | CMYGGVTEHEG |
| (SEQ ID NO:2) | (SEQ ID NO:4) |
| YGGVTEHEGN | CMYGGVTEHE |
| (SEQ ID NO:3) | (SEQ ID NO:5) |
| TEHEGN | CMYGGV |
| (SEQ ID NO:7) | (SEQ ID NO:6) |

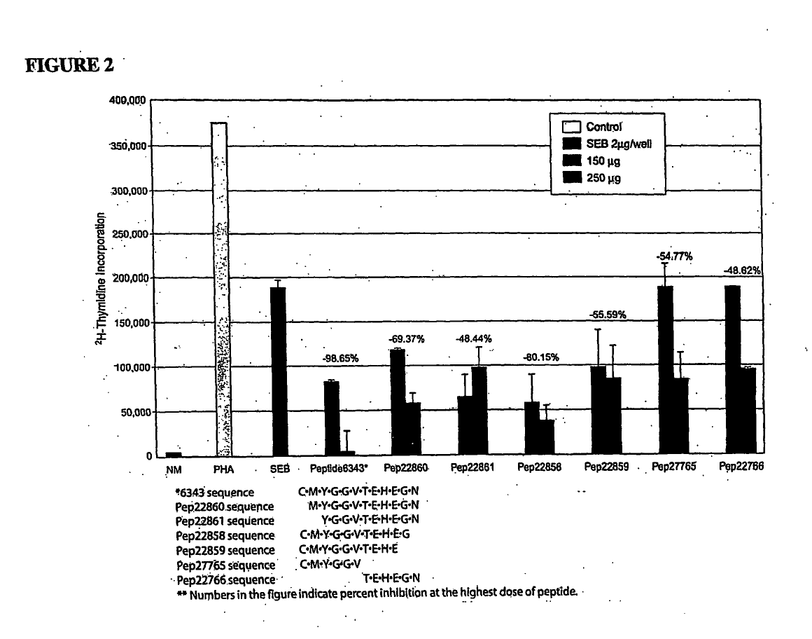

FIG. 2 shows the results of direct peptide inhibition of blastogenesis. Removal of specific amino acids from the N-terminal or C-terminal end of the original 12 amino acid peptide affects the biological properties of the peptide. As shown in FIG. 2, removal of the first amino acid from the N-terminal end of the peptide resulted in a loss of inhibiting activity of approximately 30% (98.65%-69.37%) at a dose of 250 μg. Removal of the second amino acid resulted in a loss of inhibiting activity of approximately 50%. Removal of a single amino acid from the C-terminal resulted in 18% loss of activity while removal of two amino acids had a 54% loss of activity. Finally, the use of either the first six amino acids of the N-terminal or C-terminal portion of the peptide resulted in approximately equal loss of activity of the peptide (52% and 48%, respectively). Mimetics designed to have similar properties as the peptides of the invention may act as and directly inhibit superantigen T cell stimulation.

### Example 4

### Trimer Studies

The single amino acid substitution experiments at the N-terminal portion of the molecule that completely blocked inhibitory properties of the peptide prompted peptide construction revolving around the first three N-terminus amino acids, namely C, M, and Y. FIG. 3 depicts the results of a peptide blastogenesis assay comparing the CMY trimer peptide, and its derivatives, and the original 12-mer peptide (6343) to SEB (2 μg/well). Ficoll-Hypague isolated PBMCs were mixed with varying doses of either positive control 12-mer peptide (peptide 6343) or trimer. The CMY and YMC trimers resulted in inhibiting SEB proliferation comparable to that of the original 12-mer. Acetylation of the CMY peptide did not significantly change the inhibitory effects of CMY. However, at higher doses, the D-conformation of CMY and YMC, i.e., cmy and ymc, had significantly greater inhibitory effects than the control 12-mer peptide or their respective L-conformation peptides. Low concentrations of peptides did not affect the inhibition of SEB proliferation significantly. The “scrambled” 12-mer control peptide, referred to as peptide 6343S, having an amino acid sequence of EHEGNCMYGGVT (SEQ ID NO:28), had minimal effect on bacterial toxin proliferation as demonstrated in FIG. 3.

### Example 5

### Blastogenesis Assays of Various Trimer and Dimer Peptides

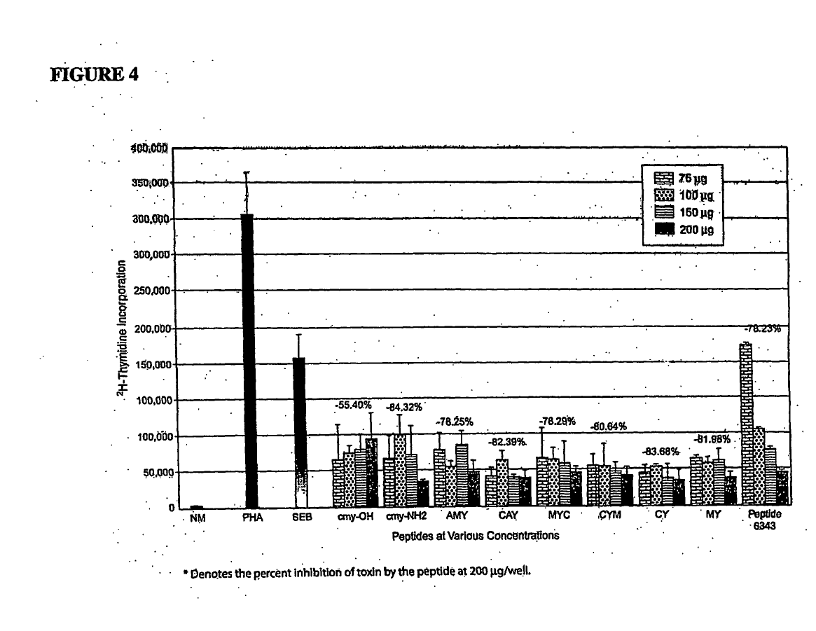

Since both the D- and L-conformations of the trimer CMY, as well as the inverted trimer peptide, YMC, were found to be effective in inhibiting the superantigen activity of the toxin, a series of trimers and dimers were constructed. Various amino acids were substituted in place of the original CMY trimer. Blastogenesis assays were performed in human PBMCs as previously described using the following peptides: cmy-OH, cmy-NH₂, AMY, CAY, MYC, CYM, CY, MY, and the original 12-mer peptide (6343). FIG. 4 demonstrates that a variety of trimers and even dimers revolving around the trimer CMY were all effective in inhibiting blastogenesis of the toxins. Upon further examination, it was also apparent that trimers not containing either cysteine (C) or methionine (M) were also effective, suggesting that tyrosine is a crucial amino acid for inhibitory activity.

### Example 6

### Blastogenesis Assays Studying the Importance of Tyrosine

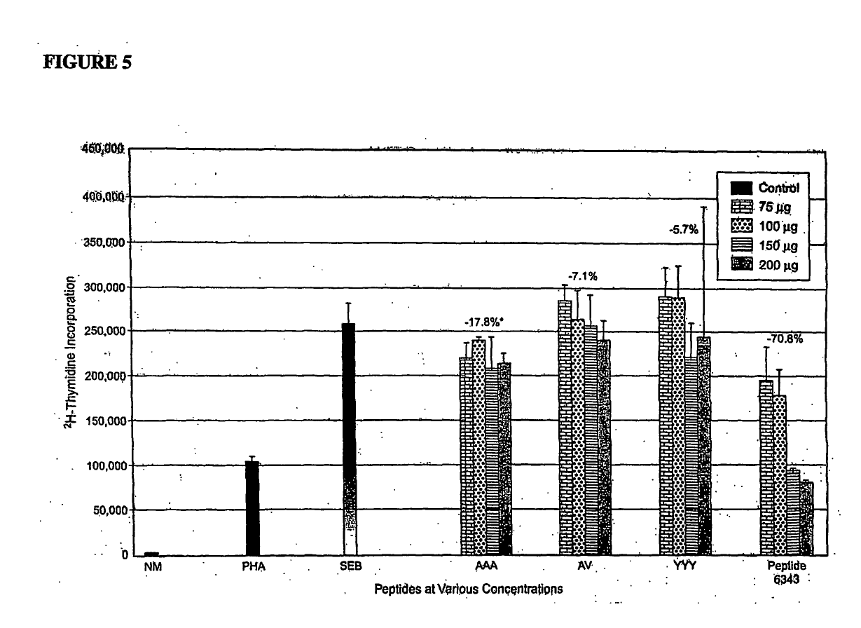

The peptides AAA, AV, and TTT were purchased from Biochem Company (King of Prussia, Pa.) and were stated to be more than 98% pure. FIG. 5 shows the results of blastogenesis assays analysing the importance of the tyrosine amino acid. The results of FIG. 5 demonstrate that either a trimer or dimer lacking tyrosine was not effective, suggesting that tyrosine is important for inhibitory activity. Surprisingly, the trimer containing three tyrosines was not inhibitory as well.

### Example 7

### Trimer and Tetrame Peptide Studies

Further trimer and tetramer peptides were constructed for analysis as follows:

| | |
|---|---|
| CMY | |
| CMYG; | (SEQ ID NO:20) |
| CMYGK; | (SEQ ID NO:21) |
| CMYKK. | (SEQ ID NO:22) |

All peptides described above were synthesized using the solid phase synthesis technique according to standard procedures (Merrifield, B., Science 232:341-347, 1986; Patarroyo, et al., Nature 328:629-632, 1987) and were prepared by Multiple Peptide Systems (San Diego, Calif.). HPLC analysis of all peptides revealed a purity of at least 95%.

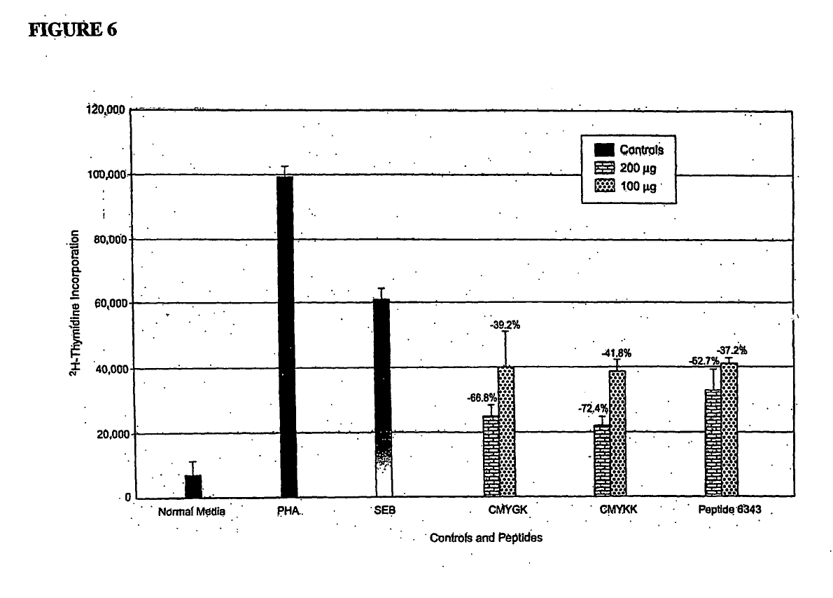

Since increased solubility enables better binding of peptides to, for example, the MHC molecule, additional amino acids which enhance solubility were added to the trimer and tetramer peptides. The addition of lysines has been reported to increase the solubility of the peptides. FIG. 6 shows the results of using peptides having added one and two lysines to the C-terminal end of the tetramer and trimer, respectively. The CMYKK (SEQ ID NO:22) peptide resulted in inhibition of SEB proliferation that was comparable and at high concentrations better than the original 12-mer peptide (6343). Similarly, the addition of a lysine to the CMYG (SEQ ID NO:20) tetramer resulted in inhibition of SEB proliferation comparable to that of the original 12 amino acid peptide. In fact, at a concentration of 200 μg, both CMYKK (SEQ ID NO:22) and CMYGK (SEQ ID NO:21) inhibited proliferation of SEB more effectively than the 12-mer peptide 6343.

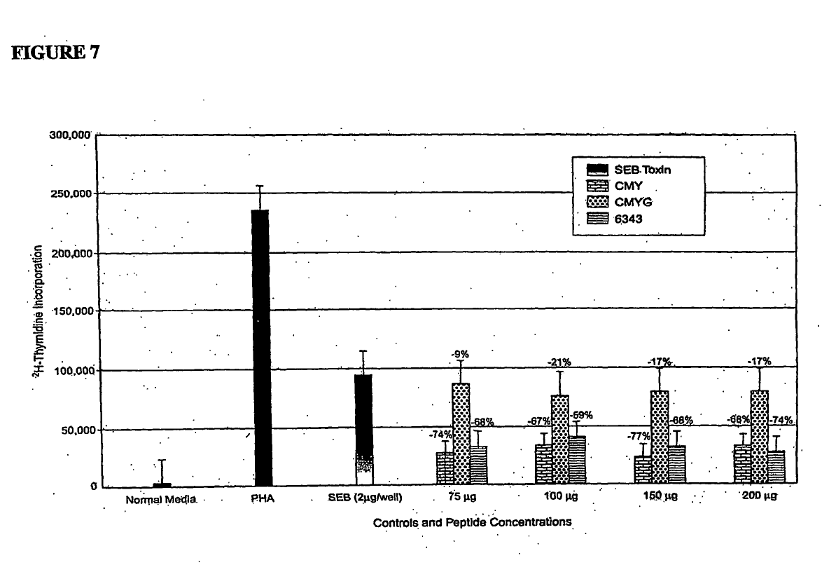

Both CMY trimer and CMYG (SEQ ID NO:20) tetramer peptides were constructed. FIG. 7 shows the average results of blastogenesis assays using these peptides. The trimer was as active as the original 12 amino acid peptide (i.e., peptide 6343) in inhibiting the proliferative effects of the toxin. However, the tetramer had comparatively little effect in the inhibition of toxin regardless of concentration.

### Example 8

### Viability Assay

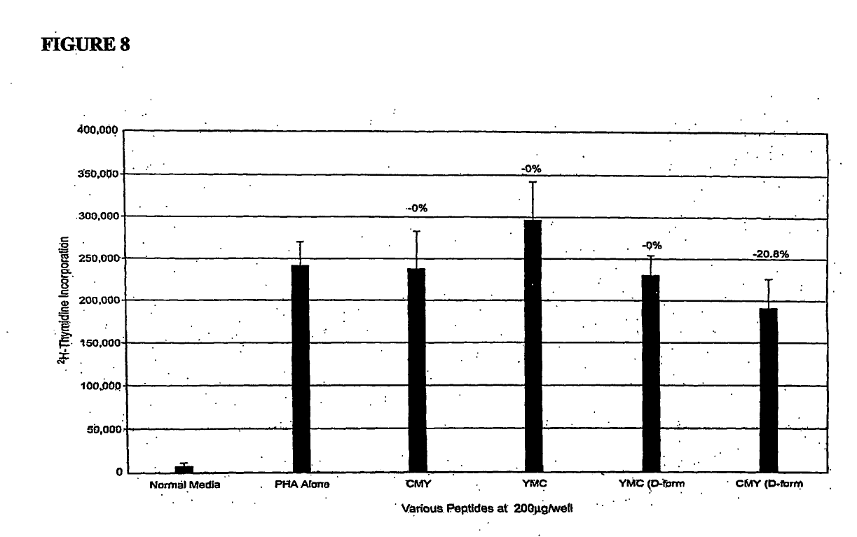

To ensure that the peptides were not interfering with normal cell function, a 72 hour phytohemagglutinin (PHA) blastogenesis assay was performed with human PBMCs (100 microliters; 2×10⁶ cells/ml) to which 200 micrograms of each peptide of interest was added to the wells of 96 well titer plates to a total volume of 200 microliters per well. All experiments were performed in triplicate. PHA, a positive mitogenic control, was added at a concentration of 5 micrograms/well. After incubating for 96 hours, 3 microCi of tritiated thymidine was added to each well and collected after an additional 18 hours incubation. The counts were analysed by a beta count reader. FIG. 8 shows that PHA stimulation was not affected even when the highest dose of each peptide (CMY, YMC, ymc, cmy) was added to the wells.

A Trypan blue exclusion assay was performed as a second viability test. Cell death for each peptide was examined over several days. Observations of PBMCs by this method revealed no difference in cell counts between cells with or without peptide.

### Example 9

### In Vivo Analysis of Mice Protected with Various Peptides

In vivo murine experiments were performed using the YMC trimer peptide in six control and four experimental BALB/c female mice (8 weeks old). Mice were first primed and sensitized with 0.1 micrograms of LPA and 20 micrograms of galactosamine per mouse via intraperitoneal injection (Blank, et al., Eur. J. Immunol. 27:825-833, 1997). Eight hours later, a suitable dose of superantigen or toxin was administered to the mice to cause approximately 100% mortality in 24 to 48 hours. In protection studies, 3.0 milligrams/100 microliters of trimer YMC peptide or saline control was administered to each animal intraperitoneally (IP) 1 hour before administration of toxin. One hour later, all mice were challenged with 0.02 micrograms SEB (via intraperitoneal injection) and the mice were observed for 24-48 hours The results are shown below in Table 3. Administration of trimer YMC prevented the induction of lethal shock in the -xperimental animals when compared to the saline injected controls.

TABLE 3

| MICE | TOTAL # MICE | # SURVIVORS | % SURVIVAL |
|---|---|---|---|
| CONTROL | 6 | 1 | 16 |
| YMC PEPTIDE | 4 | 3 | 75 |

Further studies using the D-conformation trimers, ymc* and cmy* were performed by IP injecting 8-10 weeks old female Balb/c mice at time 0 with 0.01 mgs of lipopolysaccharide (LPS; Calbiochem) and 20 mgs D-galactosamine. At 5 hours, the experimental mice received 2 mgs of the appropriate peptide IP. At 6 hours, the experimental mice were injected IP with 0.05 μgs in 100 microliters SEB (Sigma), while control mice received phosphate buffered saline (PBS) in the same volume. Table 4 shows the affects of using trimers to protect against superantigen shock in Balb/c mice.

TABLE 4

| | TOTAL # | TOXIN | PEPTIDE | SURVIVORS/ | % |
|---|---|---|---|---|---|
| MICE | MICE | (0.05 μgs) | (3 mg/DOSE) | INJECTED | SURVIVAL |
| CONTROL | 6 | SEB | 0 | 0/6 | 0 |
| EXPT 1 | 6 | SEB | YMC* | 5/6 | 83 |
| EXPT 2 | 6 | SEB | CMY* | 2/6 | 33 |

*= D-CONFORMATION TRIMER

Table 4 shows that the D-conformation ymc* trimer was quite effective in protecting mice against shock from superantigen, SEB. The D-conformation cmy* trimer also provided some protection against superantigen-induced shock.

### Example 10

### Monoclonal Superantigen Antibody Production

Two Balb/c mice were immunized with peptide 6348 (which contains both consensus regions I and II) two times at one month intervals, and was injected into two separate sites. The first injection contained 200 micrograms (200 microliters) of peptide 6348 and complete Freund's adjuvant, while the second injection used incomplete adjuvant. Retro-orbital bleedings were obtained ten days after the second injection. One more booster dose in saline was administered, and the animals were re-tested 10 days later by retro-orbital bleedings. The collected sera were tested by ELISA for antibody titers to peptide 6348. Mouse #1 had the higher titers to peptide 6348 (i.e., ₋1.0 OD, 450 nm at 1:50,000 dilution) compared to mouse #2, which had titers of 0.9 OD at 1:50,000 dilution. Mouse #1 was selected and used for further studies.

Mouse #1 was given a booster dose of 200 μg peptide 6348 IP in distilled water (100 microliters) two days before the animal was sacrificed and before fusing mouse myeloma and spleen cells. Splenocytes were obtained and treated with 84% ammonium chloride to lyse red cells according to standard protocol (Antibodies: A Laboratory Manual, 1988, Chapter 6, “Monoclonal antibodies” Eds. E. Harlow & D. Lane, Cold Spring Harbor Press). The splenocytes were then washed in Dubecco's Modified Eagle Medimn (DMEM) and resuspended and counted. The fusion was carried out using standard techniques with the mouse myeloma line SP2/0 (purchased from ATCC) at a ratio of 4 spleen cells from mouse #1 per 1 myeloma cell. The total number of splenocytes was 2×10⁷ cells in 10 ml for each 96 well plate (200,000 cells/well). The final medium was DMEM, 10% hypoxanthine-aminopterin-thymidine (HAT) solution and 10% fetal calf serum (FCS).

Culturing the fused cell mixture in HAT solution enabled the selection of myeloma/spleen fusion cells, since non-fused spleen cells had limited growth potential and died, while non-fused myeloma cells died because they cannot grow when the the De Novo nucleotide synthesis has been blocked with HAT medium. The 96 well plates were observed for two weeks at which time those wells which showed cell growth compared to control were expanded. The living and growing myeloma/spleen cell hybrids were cultured in DMEM with the addition of 5% fetal bovine serum for 20-30 days to dilute any remaining aminopterin. Usually three rounds of limiting dilutions cloning was sufficient to isolate the monoclonal cell line adequately.

Forty to fifty days after cell fusion, the supematants from the selected wells were harvested and tested for the presence of antibodies against the immunized antigen in an enzyme-linked immunosorbant assay (ELISA). The supernatents were tested in ELISA assays for activity against peptide 6348. The positive ELISA wells were transferred into 6 ml culture wells. Of the 15 clones selected, 3 clones that exhibited the highest ELISA titers against peptide 6348 were saved and prepared for further testing. The present invention provides three clones or hybridomas labeled 2D5, 1H10, and 1B1 deposited at the American Type Culture Collection (ATCC), P.O. Box 1549, Manassas, Va. 20108 on _ _ _ _ _ _ and under ATCC Accession Nos. _ _ _ _ _ _ according to the terms of the Budapest Treaty.

These 3 clones labeled 2D5, 1H10 and 1B1 were expanded for further testing using the immunoblot technique containing six superantigens that had been transferred from 15% SDS gels. The six superantigens that were tested included: SEA, SEB, SEC, SPEA, SPEC, and TSST-1. All lanes were loaded as follows: 5 μgs of toxin/well for SEA, SEB, SEC; 10 μgs of toxin/well for SPEA and TSST-1; 7 μgs of toxin/well for SPEC. The immunoblot was developed layering 2D5 monoclonal antibody for 2 hours. After removal and washes, alkaline phosphatase conjugated goat anti-mouse IgG antibody was applied for 1 hour at a dilution of 1:500. The 5-bromo-4chloro-3-indolyl phosphate/nitro blue tetrazolium (BCIP/NBT) purple liquid substrate system for membranes was added for 5 minutes and the reaction stopped with distilled water. Immunoblot experiments were similarly performed using clones 1H10 and 1B1.

The results of the immunoblot experiments for all three clones: 2D5, 1H10, and 1B1 are shown below in Table 5.

TABLE 5

| | Clone 2D5 | Clone 1H10 | Clone 1B1 |
|---|---|---|---|
| SEA | + | + | + |
| SEB | + | + | + |
| SEC | + | − | + |
| SPEA | + | − | − |
| SPEC | + | − | + |
| TSST-1 | + | − | − |

The results of the 2D5 monoclonal antibody immunoblot experiment were such that clone 2D5 showed high reactivity against the superantigens and identified 6 of the 6 superantigens tested. However, the 1H10 and 1B1 clones were not as reactive against the superantigens as that of clone 2D5. Specifically, Clone 1H10 recognized 2 superantigens: SEA and SEB, while 1B1 reacted with 4 of 6 superantigens: SEA, SEB, SEC, and SPEC.

### Example 11

### Monoclonal Superantigen Antibody Production Using Trimer Peptides

Following the method described by Mikolajczyk S D, et al. Bioconjug. Chem. 1994 Vol.5: p 636-46, periodate oxidation binds a serine to the Cysteine end of the trimer, e.g. cmy, in either the L- or D-conformation. A reductive deamination step is then performed to bind a tetanus toxoid (TT) carrier to the serine. In this manner the trimer as a hapten becomes immunogenic.

Two Balb/c mice are immunized with the trimer-TT peptide conjugate two times at one month intervals, and is injected into two separate sites. The first injection contains 200 micrograms (200 microliters) of the trimer-TT peptide and complete Freund's adjuvant, while the second injection uses incomplete adjuvant. Retro-orbital bleedings are obtained ten days after the second injection. The collected sera is tested by ELISA for the presence of antibody titers to the trimer-TT peptide. A third dose may be administered subcutaneously in saline. If Mouse #1 has higher titers to the trimer peptide compared to Mouse #2 at a 1:50,000 dilution and 450 mn, then Mouse #1 may be selected and used for further studies. Monoclonal antibodies directed to the peptides described in the instant application are produced as previously described and according to standard protocol (Antibodies: A Laboratory Manual, 1988, Chapter 6, “Monoclonal antibodies” Eds. E. Harlow & D. Lane, Cold Spring Harbor Press).

### Example 12

### Peptide Absorption studies with Monoclonal Antibodies Made Against Peptide 6348

In order to confirn that the generated monoclonal antibodies are directed against all regions of the peptide or only to consensus region 1, peptide 6348 (directed to consensus regions 1 and 2) is used for immunizing the mice and absorbing with the 12-mer peptide 6343 or fewer as described herewith. To determine whether the monoclonal antibody recognizes the whole peptide or for example, the 12-mer peptide 6343, the supernatant of clone 2D5, which recognizes all 6 superantigens, is absorbed with 10 mgs of peptide 6343 by mixing the monoclonal antibody directed against peptide 6348 at 37° C. for one hour and then mixing overnight at 4° C. After centrifugation for 30 minutes at 10,000 RPM, the supernatant is layered over the immunoblots containing the 6 superantigens. Goat anti-mouse IgG tagged with alkaline phosphatase and alkaline phosphatase substrate is used for development. The peptide 6343 antigen absorbs all of the antibodies directed against the superantigens if the monoclonal antibodies are directed to the peptide or consensus region 1. Controls include absorption with peptides unrelated to peptide 6343 as well as the consensus region 2 peptide, peptide 6346. Absorption assays are also performed with other peptides as described herein, such as, but not limited to, the L- or D-conformation trimers.

### Example 13

### Confirmation of Monoclonal Antibodies

Limiting dilutions of the selected clones, including 2D5, are performed in order to produce a clone of cells from a single fused cell which may be maintained in a cell culture and which will continue to secrete monoclonal antibodies. Expansion and testing in immunoblots are performed with the other 8 clones. The limiting dilutions are performed as follows: 10 microliters (containing 2×10⁶ cells) of each clone to be diluted is placed in 10 milliliters of DMEM with 5% FBS in such a manner that 100 cells/well are in Row 1. Then these cells are diluted 1:1 with normal medium and 100 microliters passed on to Row 2. This procedure is repeated until all 12 rows of a 96 well plate are completed. Finally, all wells receive 100 microliters of normal medium for a total of 200 microliters/well. If one of these limiting dilution clones is again positive for all six superantigens, this clone will be tested for other superantigens not yet tested, such as, but not limited to, SED, SPEG, SPEH, SPEZ. Furthermore, a second limiting dilution will be performed to ensure that a single monoclonal antibody is obtained. ELISA is performed to test for activity of the various clones. All clones, both the original and expanded clones, are retrievably frozen at −125° C. in liquid nitrogen. After purification of the clone, the monoclonal antibody is compared to the polyclonal antibody, which is already known to be protective in a mouse model of toxic shock as previously described. Positive results suggest that a single monoclonal antibody is reactive against all of the tested superantigens. This clone may be “humanized” and used in humans as a protective agent for the prevention of superantigen bioterrorism.

### Example 14

### MHC Class II Molecules Interacting with Peptide

Several 3-D models of interactions between the MHC class II molecule and the 12-mer peptide (6343) were constructed in order to determine why this 12-mer peptide was capable of inhibiting the blastogenesis properties of superantigens (Visvanathan, K., et al. Infection and Immunity 69:875-884, 2001) and the role of tyronsine in peptides that were capable of inhibiting toxin activity. Model building was accomplished using the program MODELLER (Sali, A. and Blundell, T. L. J. Mol. Biol., 234:779-815, 1993). Model building was done automatically by satisfying restraints on many distances, angles and dihedral angles using this program. The first step in comparative modeling was the identification and the analysis of the template structures, protein data bank (PDB) structures 1SEB and 2SEB (Dessen, et al. Immunity 7:473-481, 1997; Jardetzky, et al. Nature 368:711-718, 1994). The analysis of the interactions between superantigen (SEB) and the MHC class II molecule revealed that a disulfide loop (residues 92-96) contacted MHC class II molecule alpha1 (α1) domain of its alpha helix. Based on previous studies that a cysteine was involved in the disulfide loop and alpha1 domain interaction (Visvanathan, K., et al. Infection and Immunity 69:875-884, 2001), the disulfide loop was used as the template structure for modeling the interaction between the 6343 peptide and the MHC class II molecule. The first model was designed using the following target-template alignment:

| | | |
|---|---|---|
| Model 1: | | |
| Template peptide: | C--YFSKK---- | (SEQ ID NO:30) |
| Target peptide: | CMYGGVTEHEGN | (SEQ ID NO:1) |

Further analysis of the models using the program LIGPLOT (Wallace, et al. Protein Eng. 8:127-134, 1995) indicated that the tyrosine residue side chain hydrophobically interacts with residues of the MCH class II molecule, i.e., Alanine 61, Leucine 60, and Glutamine 57). This observation was consistent with the empirically observed importance of the tyrosine residue in the peptide-MHC class II interaction. Models 2 and 3 were desiged using the following target-template alignment:

| | | |
|---|---|---|
| Model 2: | | |
| Template peptide: | CYFSKK------ | (SEQ ID NO:31) |
| Target peptide: | CMYGGVTEHEGN | (SEQ ID NO:1) |
| Model 3: | | |
| Template peptide: | --CYFSKK---- | (SEQ ID NO:31) |
| Target peptide: | CMYGGVTEHEGN | (SEQ ID NO:1) |

### Example 15

### MHC Class II Binding Assay

The plates are coated with immunoaffinity purified soluble human MHC DR1 (kindly provided by Dr. Strominger, Harvard University) overnight at 4° C. in 0.1 M TRIS, pH 8.0 at a concentration of 1 μg per well. A 1% BSA solution in PBS is used to block the coated plates for 1 hour. Peptide 6343 was added to the wells at various concentrations and allowed to incubate for 1 hour. After washing in ELISA wash buffer three times, the rabbit anti-peptide antibody (6343) diluted 1:500 in RPMI was added and incubated for another hour. HRP-conjugated antibodies of appropriate affinity are used at a dilution of 1:1000. A 1:1 mixture of hydrogen peroxide and TMB substrate (100 μl; Kirekegaard and Perry, Inc.) is applied in the dark for 20 minutes after which the plate is read. All incubation steps are carried out at room temperature. Plates are washed 3 times with ELISA Wash Buffer between every incubation step. The pH of the binding medium is adjusted to ensure that all assays were at the same pH 7.0. Care is taken to ensure that the ionic strength is adjusted for in each assay. Apparent Kd, the dissociation constant at equilibrium, is calculated using Lineweaver-Burk equation (Segal, I.H. 1975. “Enzyme Kinetics”, p. 107-108. In: Behavior and Analysis of Rapid Equilibrium and Steady-State Enzyme Systems. John Wiley & Sons), as previously described by Fridkis-Hareli and J.L. Strominger (J. Immunol. 160:4386-4397, 1998).

### Example 16

### Mimetic Design

In order to design a mimetic from a compound, such as for example, but not limited to, a peptide or derivative of the invention, having a given target property, several steps are taken. First, specific components of the molecule, peptide, or derivative, which are necessary for determining the target property, are ascertained. This is accomplished by systematically varying the amino acid residues of the peptide. For example, by substituting each residue methodically, the “pharmacophore,” or active region of the compound, is determined. The tyrosine residue has been identified as a critical residue in the peptide of the invention. Having identified the pharmacophore, the structure may be modeled according to its physical properties, such as stereochemistry, bonding, size and/or charge, using techniques well known in the art. A variant method entails modeling the three-dimensional structure. A template molecule is then be chosen onto which chemical groups that mimic the pharmacophore is attached. Further, mimetics determined by this method are screened for the target property. A mimetic however, can be any molecule that mimics the structure, function, and/or actions of another molecule, including but not limited to a peptide or derivative of the invention.

Those skilled in the art will recognize, or be able to ascertain using no more than routine experimentation, many equivalents to the specific embodiments of the invention described herein. Such equivalents are intended to be encompassed by the following claims.

WHAT IS CLAMED IS:

1. A peptide or derivative thereof, which inhibits bacterial toxin activity, wherein said peptide, derivative, or mimetic comprises at least one amino acid sequence selected from the group consisting of: a contiguous methionine and tyrosine amino acid sequence; and a TEHEGN of SEQ ID NO: 7 amino acid sequence; and wherein said peptide consists essentially of 12 or fewer amino acids, with the proviso that the amino acid sequence is not an amino acid sequence which is found in any native toxin molecule and the peptide is not CMYGGVTEHEGN of SEQ ID NO: 1.

2. A peptide or derivative thereof, which inhibits bacterial toxin activity, wherein said peptide comprises an amino acid sequence selected from the group consisting of:
CY; MY; CMY; YMC; MYC; AMY; CAY; CMYGGVTEHEG of SEQ ID NO:4; CMYGGVTEHE of SEQ ID NO:5; CMYGGV of SEQ ID NO:6; TEHEGN of SEQ ID NO: 7; CMYAGVTEHEGN of SEQ ID NO:l 1; CMYGAVTEHEGN of SEQID NO:12; CMYGGATEHEGN of SEQ ID NO:13; CMYGK of SEQ ID NO:21; and CMYKK of SEQ ID NO:22.

3. The peptide or derivative thereof according to claim 1 consisting essentially of six or fewer amino acids.

4. The peptide or derivative thereof according to claim 1 consisting essentially of five or fewer amino acids.

5. The peptide or derivative thereof according to claim 1 consisting essentially of four or fewer amino acids.

6. The peptide or derivative thereof according to claim 1, wherein said peptide is a trimer.

7. The peptide or derivative, thereof according to claim 6, wherein said peptide has an amino acid sequence consisting of a trimer selected from the group consisting of: CMY, CYM, YMC, MYC, AMY, and CAY.

8. The peptide or derivative thereof according to claim 7, wherein said trimer is in a D-conformation.

9. The peptide or derivative thereof according to claim 1, wherein said peptide is a dimer.

10. The peptide or derivative thereof according to claim 9, wherein said peptide has an amino acid sequence consisting of a dimer selected from the group consisting of: CY and MY.

11. The peptide or derivative thereof according to claim 10, wherein said dimer is in a D-conformation.

12. A mimetic of a peptide or derivative according to claim 1, wherein the mimetic imitates hydrophobic interactions between an alpha1 domain alpha helix of a major histocompatibility complex class II molecule and a bacterial toxin.

13. A mimetic of a peptide or derivative according to claim 2, wherein the mimetic imitates hydrophobic interactions between an alpha1 domain alpha helix of a major histocompatibility complex class II molecule and a bacterial toxin.

14. A pharmaceutical composition comprising a peptide or derivative thereof, which inhibits bacterial toxin activity, wherein said peptide, derivative, or mimetic comprises at least one amino acid sequence selected from the group consisting of: one tyrosine residue, a contiguous methionine and tyrosine amino acid sequence, and a TEHEGN of SEQ ID NO: 7 amino acid sequence, and wherein said peptide consists essentially of 12 or fewer amino acids, with the proviso that the amino acid sequence is not an amino acid sequence which is found in any native toxin molecule and the peptide is not CMYGGVTEHEGN of SEQ ID NO: 1, and a physiologically acceptable carrier, diluent, or excipient.

15. A pharmaceutical composition comprising a peptide or derivative thereof, wherein said peptide comprises an amino acid sequence selected from the group consisting of: CY; MY; CMY; YMC; MYC; AMY; CAY; CMYGGVTEHEG of SEQ ID NO:4; CMYGGVTEHE of SEQ ID NO:5; CMYGGV of SEQ ID NO:6; TEHEGN of SEQ ID NO:7; CMYAGVTEHEGN of SEQ ID NO: 11; CMYGAVTEHEGN of SEQ ID NO: 12; CMYGGATEHEGN of SEQ ID NO: 13; CMYGK of SEQ ID NO:21; and CMYKK of SEQ ID NO:22, and a physiologically acceptable carrier, diluent, or excipient.

16. The pharmaceutical composition comprising the peptide or derivative thereof according to claim 14, consisting essentially of six or fewer amino acids, and a physiologically acceptable carrier, diluent, or excipient.

17. The pharmaceutical composition comprising the peptide or derivative thereof according to claim 14, consisting essentially of five or fewer amino acids, and a physiologically acceptable carrier, diluent, or excipient.

18. The pharmaceutical composition comprising the peptide or derivative thereof according to claim 14, consisting essentially of four or fewer amino acids, and a physiologically acceptable carrier, diluent, or excipient.

19. The pharmaceutical composition comprising the peptide or derivative thereof according to claim 14, wherein said peptide is a trimer, and a physiologically acceptable carrier, diluent, or excipient.

20. The pharmaceutical composition comprising the peptide or derivative, thereof according to claim 19, wherein said peptide has an amino acid sequence consisting of a trimer selected from the group consisting of: CMY; CYM; YMC; MYC; AMY; and CAY, and a physiologically acceptable carrier, diluent, or excipient.

21. The pharmaceutical composition comprising the peptide or derivative thereof according to claim 20, wherein said trimer is in a D-conformation, and a physiologically acceptable carrier, diluent, or excipient.

22. The pharmaceutical composition comprising the peptide or derivative thereof according to claim 14, wherein said peptide is a dimer, and a physiologically acceptable carrier, diluent, or excipient.

23. The pharmaceutical composition comprising the peptide or derivative thereof according to claim 22, wherein said peptide has an amino acid sequence consisting of any dimier selected from the group consisting of: CY and MY; and a physiologically acceptable carrier, diluent, or excipient.

24. The pharmaceutical composition comprising the peptide or derivative thereof according to claim 23, wherein said dimer is in a D-conformation, and a physiologically acceptable carrier, diluent, or excipient.

25. The pharmaceutical composition comprising a mimetic of the peptide or derivative according to claim 14, wherein the mimetic imitates hydrophobic interactions between an alpha1 domain alpha helix of a major histocompatibility complex class II molecule and a bacterial toxin, and a physiologically acceptable carrier, diluent, or excipient.

26. The pharmaceutical composition comprising a mimetic of a peptide or derivative according to claim 15, wherein the mimetic imitates hydrophobic interactions between an alpha1 domain alpha helix of a major histocompatibility complex class II molecule and a bacterial toxin, and a physiologically acceptable carrier, diluent, or excipient.

27. A method of inhibiting blastogenesis of mononuclear cells in the presence of at least one bacterial toxin comprising:
a) adding a pharmaceutically and physiologically effective amount of peptide or derivative according to claim 1 or a mimetic according to claim 12 in a physiologically acceptable diluent to mononuclear cells having at least one bacterial toxin; and
b) inhibiting blastogenesis of the mononuclear cells having at least one bacterial toxin.

28. A method of inhibiting blastogenesis of mononuclear cells in the presence of at least one bacterial toxin comprising:
a) administering to a mammal an amount of a peptide or derivative according to claim 2 or a mimetic according to claim 13; and
b) inhibiting blastogenesis.

29. The method of claim 28, wherein said mammal is a human.

30. A method of preventing or treating a mammal in need of treatnent due to toxicity of bacterial toxins comprising administering a peptide, derivative, or mimetic according to claim 1 or a mimetic according to claim 12 in a therapeutically effective amount for preventing or treating the mammal in need thereof.

31. A method of preventing or treating a mammal in need of treatment due to toxicity of bacterial toxins comprising: administering a peptide, derivative, or mimetic according to claim 2 or a mimetic according to claim 13 in a therapeutically effective amount for preventing or treating the mammal in need thereof.

32. A method of inducing antibodies that bind at least one bacterial toxin comprising:
a) administering to a mammal an amount of a peptide, derivative, or mimetic according to claim 1 or a mimetic according to claim 12, conjugated to a physiologically acceptable carrier, and
b) eliciting the production of said antibodies that bind at least one bacterial toxin.

33. A method of inducing antibodies that bind at least one bacterial toxin comprising:
a) administering to a mammal an amount of a peptide, derivative, or mimetic according to claim 2 or a mimetic according to claim 13, conjugated to a physiologically acceptable carrier; and
b) eliciting the production of said antibodies that bind at least one bacterial toxin.

34. A method of passively immunizing a mammal against the toxic effects of bacterial toxins comprising administering in vivo an immunologically sufficient amount of a pharmaceutical composition comprising an antibody which binds to a peptide, derivative, or mimetic according to claim 1 or a mimetic according to claim 12 and wherein said antibody binds to at least one bacterial toxin.

35. A method of passively immunizing a mammal against the toxic effects of bacterial toxins comprising administering in vivo an immunologically sufficient amount of a pharmaceutical composition comprising an antibody which binds to a peptide, derivative, or mimetic according to claim 2 or a mimetic according to claim 13 and wherein said antibody binds to at least one bacterial toxin.

36. A nucleic acid encoding at least one peptide or derivative of claim 1.

37. A nucleic acid encoding at least one peptide, derivative, or mimetic of claim 2.

38. A host cell containing the nucleic acid of claim 36.

39. A host cell containing the nucleic acid of claim 37.

40. A method of inducing antibodies that bind bacterial toxins comprising:
a) administering to a mammal in need a nucleic acid of claim 36 in a physiologically acceptable carrier;
b) expressing an immunologically sufficient amount of the encoded peptide; and
c) eliciting said antibodies.

41. A method of inducing antibodies that bind bacterial toxins comprising:
a) administering to a mammal in need a nucleic acid of claim 37, in a physiologically acceptable carrier;
b) expressing an immunologically sufficient amount of the encoded peptide; and
c) eliciting said antibodies.

42. A method of detecting the presence of antibodies to bacterial toxins in a sample comprising:
a) contacting said sample with a peptide, derivative, or mimetic of claim 1 or a mimetic of claim 12; and
b) detecting the peptide or derivative bound to said antibodies.

43. A method of detecting the presence of antibodies to bacterial toxins in a sample comprising:
a) contacting said sample with a peptide, derivative, or mimetic of claim 2 or a mimetic of claim 13; and
b) detecting the peptide or derivative bound to said antibodies.

44. An antibody made by the method of claim 40.

45. An antibody made by the method of claim 41.

46. A kit for detecting the presence of bacterial toxins in a sample comprising contacting said sample with any one of the antibodies of claim 44 and detecting the antibody bound to said toxin.

47. A kit for detecting the presence of bacterial toxins in a sample comprising contacting said sample with any one of the antibodies of claim 45 and detecting the antibody bound to said toxin.

48. A peptide, derivative thereof, or mimetic, which inhibits bacterial toxin activity, consisting essentially of an amino acid sequence of CMY.

49. A peptide, derivative thereof, or mimetic, which inhibits bacterial toxin activity, consisting essentially of an amino acid sequence of YMC.

50. A peptide, derivative thereof, or mimetic, which inhibits bacterial toxin activity, consisting essentially pf an amino acid sequence of CAY.

51. A peptide, derivative thereof, or mimetic, which inhibits bacterial toxin activity, consisting essentially of an amino acid sequence of AMY.

52. A peptide, derivative thereof, or mimetic, which inhibits bacterial toxin activity, consisting essentially of an amino acid sequence of MYC.

53. A peptide, derivative thereof, or mimetic, which inhibits bacterial toxin activity, consisting essentially of an amino acid sequence of CYM.

54. A peptide, derivative thereof, or mimetic, which inhibits bacterial toxin activity, consisting essentially of an amino acid sequence of cmy, wherein the peptide is a D-conformation peptide.

55. A peptide, derivative thereof, or mimetic, which inhibits bacterial toxin activity, consisting essentially of an amino acid sequence of ymc, wherein the peptide is a D-conformation peptide.

56. A peptide, derivative thereof, or mimetic, which inhibits bacterial toxin activity, consisting essentially of an amino acid sequence of CY.

57. A peptide, derivative thereof, or mimetic, which inhibits bacterial toxin activity, consisting essentially of an amino acid sequence of MY.

58. A peptide, derivative thereof, or mimetic, which inhibits bacterial toxin activity, consisting essentially of an amino acid sequence of CMYAGVTEHEGN of SEQ ID NO: 11.

59. A peptide, derivative thereof, or mimetic, which inhibits bacterial toxin activity, consisting essentially of an amino acid sequence of CMYGAVTEHEGN of SEQID NO: 12.

60. A peptide, derivative thereof, or mimetic, which inhibits bacterial toxin activity, consisting essentially of an amino acid sequence of CMYGGATEHEGN of SEQ ID NO:13.

61. The peptide or derivative thereof of any one of claims 1-11 and claims 48-60, wherein said peptide or derivative thereof is a peptide.

62. The antibody of claim 44, wherein said antibody is a monoclonal antibody.

63. The antibody of claim 45, wherein said antibody is a monoclonal antibody.

64. A hybridoma which produces the monoclonal antibody of claim 62.

65. A hybridoma which produces the monoclonal antibody of claim 63.


SEQUENCE LISTING (31 sequences)

SEQ ID NO: 1 - protein, 12 aa, artificial
  Synthetic Peptide
CMYGGVTEHEGN

SEQ ID NO: 2 - protein, 11 aa, artificial
  Synthetic Peptide
MYGGVTEHEGN

SEQ ID NO: 3 - protein, 10 aa, artificial
  Synthetic Peptide
YGGVTEHEGN

SEQ ID NO: 4 - protein, 11 aa, artificial
  Synthetic Peptide
CMYGGVTEHEG

SEQ ID NO: 5 - protein, 10 aa, artificial
  Synthetic Peptide
CMYGGVTEHE

SEQ ID NO: 6 - protein, 6 aa, artificial
  Synthetic Peptide
CMYGGV

SEQ ID NO: 7 - protein, 6 aa, artificial
  Synthetic Peptide
TEHEGN

SEQ ID NO: 8 - protein, 12 aa, artificial
  Synthetic Peptide
AMYGGVTEHEGN

SEQ ID NO: 9 - protein, 12 aa, artificial
  Synthetic Peptide
CAYGGVTEHEGN

SEQ ID NO: 10 - protein, 12 aa, artificial
  Synthetic Peptide
CMAGGVTEHEGN

SEQ ID NO: 11 - protein, 12 aa, artificial
  Synthetic Peptide
CMYAGVTEHEGN

SEQ ID NO: 12 - protein, 12 aa, artificial
  Synthetic Peptide
CMYGAVTEHEGN

SEQ ID NO: 13 - protein, 12 aa, artificial
  Synthetic Peptide
CMYGGATEHEGN

SEQ ID NO: 14 - protein, 12 aa, artificial
  Synthetic Peptide
CMYGGVAEHEGN

SEQ ID NO: 15 - protein, 12 aa, artificial
  Synthetic Peptide
CMYGGVTAHEGN

SEQ ID NO: 16 - protein, 12 aa, artificial
  Synthetic Peptide
CMYGGVTEAEGN

SEQ ID NO: 17 - protein, 12 aa, artificial
  Synthetic Peptide
CMYGGVTEHAGN

SEQ ID NO: 18 - protein, 12 aa, artificial
  Synthetic Peptide
CMYGGVTEHEAN

SEQ ID NO: 19 - protein, 12 aa, artificial
  Synthetic Peptide
CMYGGVTEHEGA

SEQ ID NO: 20 - protein, 4 aa, artificial
  Synthetic Peptide
CMYG

SEQ ID NO: 21 - protein, 5 aa, artificial
  Synthetic Peptide
CMYGK

SEQ ID NO: 22 - protein, 5 aa, artificial
  Synthetic Peptide
CMYKK

SEQ ID NO: 23 - protein, 14 aa, artificial
  Synthetic Peptide
CMYGGVTEHEGNGC

SEQ ID NO: 24 - protein, 28 aa, artificial
  Synthetic Peptide
CGKKNVTVQELDYKIRKYLVDNKKLYGC

SEQ ID NO: 25 - protein, 38 aa, artificial
  Synthetic Peptide
CMYGGVTEHEGNKKNVTVQELDYKIRKYLVDNKKLYGC

SEQ ID NO: 26 - protein, 12 aa, artificial
  Synthetic Peptide
YNKKKATVQELD

SEQ ID NO: 27 - protein, 12 aa, Staphylococcus aureus
CMYGGVTEHNGN

SEQ ID NO: 28 - protein, 12 aa, artificial
  Synthetic Peptide
EHEGNCMYGGVT

SEQ ID NO: 29 - protein, 12 aa, artificial
  Synthetic Peptide
TNKKKVTAQELD

SEQ ID NO: 30 - protein, 5 aa, artificial
  Synthetic Peptide
YFSKK

SEQ ID NO: 31 - protein, 6 aa, artificial
  Synthetic Peptide
CYFSKK